(19) United States
(12) Patent Application Publication
(10) Pub. No.: US 2015/0376703 A1
(43) Pub. Date: Dec. 31, 2015

(54) METHOD AND SYSTEM TO PREDICT RESPONSE TO PAIN TREATMENTS

(71) Applicant: PATHWAY GENOMICS CORPORATION, San Diego, CA (US)

(72) Inventors: Andria Del Tredici, San Diego, CA (US); Russell Kuo-fu Chan, San Diego, CA (US); Guangdan Zhu, San Diego, CA (US); K. David Becker, San Diego, CA (US); Tanya Moreno, San Diego, CA (US)

(21) Appl. No.: 14/769,409

(22) PCT Filed: Mar. 12, 2014
(86) PCT No.: PCT/US14/24939

§ 371 Date: Aug. 20, 2015

Related U.S. Application Data
(63) Continuation-in-part of application No. 13/917,509, filed on Jun. 13, 2013.
(60) Provisional application No. 61/800,560, filed on Mar. 15, 2013.
(60) Provisional application No. 61/800,506, filed on Mar. 15, 2013.

(51) Int. Cl. C12Q 1/68; G06F 19/20

(52) U.S. Cl. CPC C12Q 1/6883; G06F 19/20; C12Q 2600/106; C12Q 2600/158

(57) ABSTRACT

The present invention relates to systems and methods for predicting an individuals likely response to a pain medication comprising genotyping genetic variations in an individual to determine the individual's propensity for metabolizing a pain medication and likely response to a medication, and preferably diverse reactions to a medication. In particular, the invention comprises analyzing a biological sample provided by an individual, typically a patient or an individual diagnosed with a particular disorder, determining the individual's likely response to a particular treatment, more specifically a pain medication, and thereafter displaying, or further, recommending a plan of action or inaction. In particular, the present invention provides a grading method and system to profile an individual's response to one or more pain medication.

### RELATED APPLICATIONS

The present application claims priority to U.S. Provisional Application Ser. No. 61/800,506, “Method And System To Predict Response To Pain Treatments” filed Mar. 15, 2013, the contents of which are hereby incorporated by reference in their entirety. The present application also claims priority to U.S. Provisional Application Ser. No. 61/800,560, “Method And System To Predict Response To Pain Treatments” filed Mar. 15, 2013, the contents of which are hereby incorporated by reference in their entirety.

### FIELD OF THE INVENTION

The invention relates to methods and assays to predict the response of an individual to an analgesic treatment and to a method to improve medical treatment of a disorder, which is responsive to treatment with a pain treatment.

### BACKGROUND OF THE INVENTION

Pain of any type is the most frequent reason for physician consultation in the United States, prompting half of all Americans to seek medical care annually. It is a major symptom in many medical conditions, significantly interfering with a person's quality of life and general functioning. Diagnosis is based on characterizing pain in various ways, according to duration, intensity, type (dull, burning or stabbing), source, or location in body. Usually pain stops without treatment or responds to simple measures such as resting or taking an analgesic, and it is then called acute pain. But it may also become intractable and develop into a condition called chronic pain, in which pain is no longer considered a symptom but an illness by itself.

Pain can be classified according to many schemes and circumstances. There are two basic types of pain: acute and chronic. Acute pain occurs for brief periods of time and is associated with temporary disorders. However, it is always an alarm signal that something may be wrong. Chronic pain is continuous and recurrent. It is associated with chronic diseases and is one of their symptoms. Pain intensity not only depends on the type of stimulus that caused it, but also on the subjective perception of the pain. Despite a wide range of subjective perception, several types of pain have been classified according to:
The stimulus that caused the pain.
The pain's duration.
The features of pain (intensity, location, etc.).

Another classification system is as follows:
Gnawing pain. Continuous with constant intensity. It generally worsens with movement.
Throbbing pain. This is typical of migraine pain. It is caused by dilation and constriction of the cerebral blood vessels.
Stabbing pain. Intense and severe. It is caused by mechanical stimuli.
Burning pain. A constant, burning feeling, like, for example, the type of pain caused by heartburn.
Pressing pain. Caused by constriction of the blood vessels or muscles.

There are also specific types of pain:
Muscle pain. Also known as myalgia, this pain involves the muscles and occurs after excessive exertion or during inflammation.
Colicky pain. Caused by muscle contractions of certain organs, such as the uterus during the menstrual period. Generally cyclic in nature.
Referred pain. Occurs when the painful sensation is felt in a site other than the one where it is actually occurring, depending upon how the brain interprets information it receives from the body.
Post-surgical or Post-operative pain. Occurs after surgery and is due to lesions from surgical procedures.
Bone cancer pain. Certain types of cancers, such as prostate, breast, or other soft-tissue tumors, may progress to a painful disorder of the bone known as metastatic bone disease.

The genetic make-up of a person can contribute to the individually different responses of persons to a medicine (Roses, Nature 405:857-865, 2000). Examples of genetic factors, which determine drug tolerance, are drug allergies and severely reduced metabolism due to genetic absence of suitable enzymes. A case of a lethal lack of metabolism due to cytochrome P-450 2D6 genetic deficiency is reported by Sallee et at J Child & Adolesc. Psychopharmacol, 10: 27-34, 2000. The metabolic enzymes in the liver occur in polymorphic variants, causing some persons to metabolize certain drugs slowly and making them at risk for side effects due to excessively high plasma drug levels.

Both published literature studies and clinical experience reveal great variability in an individual's response to drug treatment with regard to drug metabolism, side effects and efficacy.

### SUMMARY OF THE INVENTION

The invention is related to methods and systems to the present invention for predicting an individual's likely response to a pain medication comprising genotyping or sequencing genetic variations in an individual to determine the individual's propensity for 1) metabolizing a pain medication and 2) likely response to a medication, and preferably 3) adverse reaction to a medication; and the software and algorithms to analyze the genetic information. In particular, the invention comprises analyzing a biological sample provided by an individual, typically a patient or an individual diagnosed with a particular disorder, determining the individual's likely response to a particular treatment, more specifically a pain medication, and thereafter displaying, or further, recommending a plan of action or inaction. In particular, the present invention provides a grading method and system to profile an individual's response to one or more pain medication. In an alternate embodiment, the present invention is directed to a method and system to recommend pain medications suitable for the individual.

These methods to identify gene mutation variants are not limited by the technique that is used to identify the mutation of the gene of interest. Methods for measuring gene mutations are well known in the art and include, but are not limited to, immunological assays, nuclease protection assays, northern blots, in situ hybridization, Polymerase Chain Reaction (PCR) such as reverse transcriptase Polymerase Chain Reaction (RT-PCR) or Real-Time Polymerase Chain Reaction, expressed sequence tag (EST) sequencing, cDNA microarray hybridization or gene chip analysis, subtractive cloning, Serial Analysis of Gene Expression (SAGE), Massively Parallel Signature Sequencing (MPSS), and Sequencing-By-Synthesis (SBS).

After a patient has been identified as likely to be responsive to the therapy based on the identity of one or more of the genetic markers identified herein, the method may further comprise administering or delivering an effective amount of a pain treatment or an alternative treatment, to the patient, based on the outcome of the determination. Methods of administration of pharmaceuticals and biologicals are known in the art and are incorporated herein by reference.

It is conceivable that one of skill in the art will be able to analyze and identify genetic markers in situ at some point in the future. Accordingly, the inventions of this application are not to be limited to requiring isolation of the genetic material prior to analysis.

These methods also are not limited by the technique that is used to identify the polymorphism of interest. Suitable methods include but are not limited to the use of hybridization probes, antibodies, primers for PCR analysis, and gene chips, slides and software for high throughput analysis. Additional genetic markers can be assayed and used as negative controls.

This invention also provides a panel, kit, gene chip and software for patient sampling and performance of the methods of this invention. The kits contain gene chips, slides, software, probes or primers that can be used to amplify and/or for determining the molecular structure, mutations or expression level of the genetic markers identified above. Instructions for using the materials to carry out the methods are further provided.

This invention also provides for a panel of genetic markers selected from, but not limited to the genetic polymorphisms identified herein or in combination with each other. The panel comprises probes or primers that can be used to amplify and/or for determining the molecular structure of the polymorphisms identified above. The probes or primers can be used for all RT-PCR methods as well as by a solid phase support such as, but not limited to a gene chip or microarray. The probes or primers can be detectably labeled. This aspect of the invention is a means to identify the genotype of a patient sample for the genes of interest identified above.

The disclosure may be further understood through reference to the following numbered embodiments.

1. A method for predicting an individual's likely response to a pain medication, comprising genotyping genetic variations in an individual to determine.
a categorical grade to an individual's likely ability to metabolize a particular pain medication and a categorical grade for a pain medication's potential efficacy with respect to the individual;
aggregating the categorical grades; and thereafter
identifying the least positive grade as the recommended prediction for the individual.

2. The method of embodiment 1, further comprising genotyping genetic variations in the individual to determine a categorical grade for the individual to have a negative adverse reaction to the particular pain medication.

3. The method of any one of embodiments 1-2, wherein the pain medication is for chronic pain.

4. The method of any one of embodiments 1-3, wherein a genetic variation in the individual will reassign one or more of the categorical grades from a default category of typical use to preferential use or precautionary use.

5. The method of any one of embodiments 1-4, wherein a drug is prescribed to the individual with a recommendation of:

Use as directed

Preferential Use

Precautionary Use

6. The method of any one of embodiments 1-4, wherein each categorical grade is assigned to the three or more categories below:

Use as Directed

Preferential Use

May Have Limitations or Significant Limitations

May Cause Serious Adverse Events

7. The method of any one of embodiments 1-6, wherein the medication is a pain medication selected from acetaminophen, non-steroidal anti-inflammatory drug, corticosteroid, narcotic, or anti-convulsant.

8. The method of any one of embodiments 1-7, wherein the medication is a narcotic.

9. The method of any one of embodiments 1-8, wherein the narcotic is an opioid, opiate or opiate derivative.

10. The method of any one of embodiments 1-9, wherein the narcotic is selected from alfentanil, alphaprodine, anileridine, bezitramide, buprenorphine, butorphanol, codeine, dezocine, dihydrocodeine, diphenoxylate, ethylmorphine, fentanyl, heroin, hydrocodone, hydromorphone, isomethadone, levomethorphan, levorphanol, meptazinol, metazocine, metopon, morphine, nalbuphine, nalmefene, opium extracts, opium fluid extracts, pentazocine, propoxyphene, powdered opium, granulated opium, raw opium, tincture of opium, oxycodone, oxymorphone, pethidine(meperidine), phenazocine, piminodine, racemic methadone, racemethorphan, racemorphan, sufentanil, thebaine, or tramadol.

11. The method of any one of embodiments 1-10, wherein said method comprises genotyping a panel of at least one gene that affects the rate of drug metabolism and a panel of genes that affect a medication's potential efficacy with respect to the individual,

12. The method of any one of embodiments 1-11, wherein said method further comprises genotyping a panel of genes that affect the propensity for the individual to have a negative adverse reaction to a particular medication.

13. The method of any one of embodiments 1-12, wherein the panel for affecting drug metabolism comprises at least one gene that affects biochemical modification of pharmaceutical substances or xenobiotics and the panel for affecting efficacy comprises at least one opioid receptor modulating gene.

14. The method of any one of embodiments 1-13, wherein the panel for affecting adverse effect comprises at least one gene for undesired effects, e.g., side effects, that can be categorized as 1) mechanism based reactions and 2) idiosyncratic, “unpredictable” effects apparently unrelated to the primary pharmacologic action of the compound.

15. The method of any one of embodiments 1-14, wherein the panel of genes for affecting metabolism is at least one cytochrome P450 gene,

16. The method of any one of claims 1-15, wherein the panel for genes for affecting metabolism is at least two cytochrome P450 genes.

17. The method of any one of embodiments 1-16, wherein the panel of genes for affecting metabolism is at least one gene selected from CYP1A1, CYP2A6, CYP2C9, CYP2D6, CYP2E1, CYP3A5, CYP1A2, CYP1B1, CYP2B6, CYP2C8, CYP2C18, CYP2C19, CYP2E1, CYP3A4, CYP3A5, UGT1A4, UGT1A1, UGT1A9, UGT2B4, UGT2B7, UGT2B15, NAT1, NAT2, EPHX1, MTHFR, and ABCB1.

18. The method any one of embodiments 1-17, wherein the panel of genes for affecting efficacy is at least one gene for an opioid receptor gene.

19. The method of any one of embodiments 1-18, wherein the panel of genes for affecting efficacy a mu-opioid receptor gene.

20. The method of any one of embodiments 1-19, wherein the panel of genes for affecting drug metabolism is CYP2D6 and CYP2B6 genes, and wherein the panel of genes for affecting efficacy is the opioid receptor gene (OPRM1).

21. The method of any one of embodiments 1-20, wherein the panel of genes for affecting adverse reactions is selected from the serotonin receptor 2A (HTR2A), the serotonin gene 2C (HTR2C) and the major histocompatibility complex, class I, B (HLA-B).

22. The method of any one of embodiments 1-21, further comprising detecting a single nucleotide polymorphism in a gene of interest within each panel.

23. The method according to any one of embodiments 1-22, wherein said genotyping comprises analyzing a sample from the individual.

24. The method according to any one of embodiments 1-23, wherein said samples is selected from blood, including serum, lymphocytes, lymphoblastoid cells, fibroblasts, platelets, mononuclear cells or other blood cells, from saliva, liver, kidney, pancreas or heart, urine or from any other tissue, fluid, cell or cell line derived from the human body.

25. A computerized system for predicting an individual's likely response to a pain medication, comprising accessing the individual's genotype information, and
determining a categorical grade to an individual's likely ability to metabolize a particular pain medication and a categorical grade for a pain medication's potential efficacy with respect to the individual;
aggregating the categorical grades; and thereafter
identifying the least positive grade as the recommended prediction for the individual.

26. The computerized system of embodiment 25, wherein the system is accessed by healthcare providers.

27. The computerized system of any one of embodiments 25-26, wherein any potential conflicts and problems are flagged and displayed for the provider to review.

28. The computerized system of any one of embodiments 25-27, wherein a report is generated displaying recommendations for one or more medications.

29. The computerized system of any one of embodiments 25-28, wherein a genetic variation in the individual will reassign one or more of the categorical grades from a default category of typical use to preferential use or precautionary use.

30. The computerized system of any one of embodiments 25-29, wherein the pain medications is selected from acetaminophen, non-steroidal anti-inflammatory drug, corticosteroid, narcotic, or anti-convulsant.

31. The computerized system of any one of embodiments 25-30, wherein said genotyped information comprises a panel of at least one gene that affects the rate of drug metabolism and a panel of genes that affect a pain medication's potential efficacy with respect to the individual.

32. The computerized system of any one of embodiments 25-31, wherein said genotyped information further comprises a panel of genes that affect the propensity for the individual to have a negative adverse reaction to the particular pain medication.

33. A method of advising patient pain drug selection comprising the steps of identifying a patent having a pain symptom to be addressed pharmaceutically, identifying at least a drug to pharmaceutically address said pain symptom, assaying genomic information of said patient, evaluating the efficacy of said drug in view of said genetic information of said patient, and providing to said patient a report evaluating said efficacy.

34. The method of embodiment 33 wherein said symptom is a symptom listed in FIG. 8.

35. The method of any one of embodiments 33-34 wherein said drug is a drug listed in FIG. 8.

36. The method of any one of embodiments 33-35 wherein said efficacy is an efficacy listed in FIG. 8.

37. The method of any one of embodiments 33-36 wherein said evaluating comprises placing a drug into a category.

38. The method of any one of embodiments 33-37, wherein said categorizing comprises placing said drug into one of four categories related to drug efficacy in view of patient genomic information.

39. The method of any one of embodiments 33-38, wherein said placing said drug into one of four categories comprises describing a drug as having ‘preferential use’,′ use as directed,′ significant limitations,′ or ‘serious adverse events.’

40. The method of any of embodiments 33-39, further comprising subjecting said report to a medical doctor's review prior to providing to said patient.

### BRIEF DESCRIPTION OF THE DRAWINGS

FIG. 1 displays the interaction of an individual and his caregiver in the system.

FIG. 2 describes the mechanism for providing warnings or recommendations to particular pain treatments based on the efficacy of a particular treatment balanced against any potential conflicts or problems as they relate to the genotype of an individual.

FIG. 3. describes the process for a caregiver in interacting with the system.

FIG. 4 is an illustration of data stores accessed to generate a recommendation for treatments.

FIG. 5 is an illustration of a of a computer system that can perform the methods of the invention.

FIG. 6 is a diagram illustrating portals for interacting with the system for an individual (or their caregiver).

FIG. 7 is a simplified example of the output of the algorithm with the recommendation categories for all tested drugs

FIG. 8 is a sample output of the algorithm with the recommendation categories for all tested drugs and a text for each drug that is not assigned to the “Use as Directed” category. The text includes detailed reasons for the category assignment and, when appropriate, clinical recommendations.

### DETAILED DESCRIPTION OF THE PREFERRED EMBODIMENTS

Before the compositions and methods are described, it is to be understood that the invention is not limited to the particular methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

Throughout this disclosure, various publications, patents and published patent specifications are referenced by an identifying citation. The disclosures of these publications, patents and published patent specifications are hereby incorporated by reference in their entirety into the present disclosure to more fully describe the state of the art to which this invention pertains.

### DEFINITIONS

The term “disease state” is used herein to mean a biological state where one or more biological processes are related to the cause or the clinical signs of the disease. For example, a disease state can be the state of a diseased cell, a diseased organ, a diseased tissue, or a diseased multi-cellular organism. Such diseases can include, for example, pain which affects the entire population at one time or another, can be either or both chronic and acute. Although pain is most often a symptom of a disorder, it can also be a disorder in and of itself. Spinal injuries are most closely associated with chronic pain, but other disorders, such as systemic infections, arthritis and cancer, are also causes of chronic pain. The treatment of pain, including chronic pain, typically involves the administration of analgesic medication. Analgesics relieve pain by altering a patient's perception of nociceptive stimuli without producing anesthesia or loss of consciousness. Although there have been some efforts to find objective indicators for pain, those efforts are hampered by the problems of genetic variability and variations due to an individual's perception of pain. One study provided an objective diagnostic test for peripheral nerve damage that causes chronic spinal pain. U.S. Pat. No. 5,364,793 and U.S. Pat. No. 5,583,201, both of which are specifically incorporated by reference, describe an acute phase protein, apolipoprotein E, originally thought to correlate with damage caused by peripheral nerve damage which caused chronic spinal pain (Vanderputten D. M. et al., Applied Theoretical Electrophoresis, 3:247-252, 1993). it was later found that this correlation was not statistically significant for clinical use. Thus, it is still very difficult to accurately and objectively assess another person's pain level. Consequently, determining the correct medication and determining the proper dosage of that medication to treat a patient's pain is equally difficult.

The present invention is directed to treating all types of pain. In particular, acute, subacute, and chronic pain is included. Specific types of chronic pain include neuropathic, somatic, and visceral pain.

Clinically, pain can be classified temporally as acute, subacute, or chronic; quantitatively as mild, moderate, or severe; physiologically as somatic, visceral, or neuropathic; and etiologically as medical or psychogenic. Acute pain (such as postoperative pain or acute traumatic pain) typically has objective signs and associated autonomic nervous system hyperactivity with tachycardia, hypertension, and diaphoresis being present. Chronic pain occurs for periods of time for three months or longer on a recurring basis. The quantitative nature (i.e. intensity) of the pain is the major factor in choosing drug therapy. These conditions include, but are not limited to, chronic pain conditions, fibromyalgia syndrome, tension headache, migraine headache, phantom limb sensations, irritable bowel syndrome, chronic lower back pain, chronic fatigue, multiple chemical sensitivities, temporomandibular joint disorder, post-traumatic stress disorder, chronic idiopathic pelvic pain, Gulf War Syndrome, vulvar vestibulitis, osteoarthritis, rheumatoid arthritis, angina pectoris, postoperative pain (e.g., acute postoperative pain), and neuropathic pain. In general, these conditions are characterized by a state of pain amplification as well as psychosocial distress, which is characterized by high levels of somatization, depression, anxiety and perceived stress.

Neuropathic pain is a common variety of chronic pain. It can be defined as pain that results form an abnormal functioning of the peripheral and/or central nervous system. A critical component of this abnormal functioning is an exaggerated response of pain-related nerve cells either in the periphery or in the central nervous system. Somatic pain results from activation of peripheral receptors and somatic sensory efferent nerves, without injury to the peripheral nerve or CNS. Visceral pain results from visceral nociceptive receptors and visceral efferent nerves being activated and is characterized by deep, aching, cramping sensation often referred to cutaneous sites.

An “agonist” refers to an agent that binds to a polypeptide or polynucleotide of the invention, stimulates, increases, activates, facilitates, enhances activation, sensitizes or up regulates the activity or expression of a polypeptide or polynucleotide of the invention.

An “antagonist” refers to an agent that inhibits expression of a polypeptide or polynucleotide of the invention or binds to, partially or totally blocks stimulation, decreases, prevents, delays activation, inactivates, desensitizes, or down regulates the activity of a polypeptide or polynucleotide of the invention.

“Inhibitors,” “activators,” and “modulators” of expression or of activity are used to refer to inhibitory, activating, or modulating molecules, respectively, identified using in vitro and in vivo assays for expression or activity, e.g., ligands, agonists, antagonists, and their homologs and mimetics. The term “modulator” includes inhibitors and activators. Inhibitors are agents that, e.g., inhibit expression of a polypeptide or polynucleotide of the invention or bind to, partially or totally block stimulation or enzymatic activity, decrease, prevent, delay activation, inactivate, desensitize, or down regulate the activity of a polypeptide or polynucleotide of the invention, e.g., antagonists. Activators are agents that, e.g., induce or activate the expression of a polypeptide or polynucleotide of the invention or bind to, stimulate, increase, open, activate, facilitate, enhance activation or enzymatic activity, sensitize or up regulate the activity of a polypeptide or polynucleotide of the invention, e.g., agonists. Modulators include naturally occurring and synthetic ligands, antagonists, agonists, small chemical molecules and the like. Assays to identify inhibitors and activators include, e.g., applying putative modulator compounds to cells, in the presence or absence of a polypeptide or polynucleotide of the invention and then determining the functional effects on a polypeptide or polynucleotide of the invention activity. Samples or assays comprising a polypeptide or polynucleotide of the invention that are treated with a potential activator, inhibitor, or modulator are compared to control samples without the inhibitor, activator, or modulator to examine the extent of effect. Control samples (untreated with modulators) are assigned a relative activity value of 100% Inhibition is achieved when the activity value of a polypeptide or polynucleotide of the invention relative to the control is about 80%, optionally 50% or 25-1%. Activation is achieved when the activity value of a polypeptide or polynucleotide of the invention relative to the control is 110%, optionally 150%, optionally 200-500%, or 1000-3000% higher.

The term “test compound” or “drug candidate” or “modulator” or grammatical equivalents as used herein describes any molecule, either naturally occurring or synthetic, e.g., protein, oligopeptide (e.g., from about 5 to about 25 amino acids in length, preferably from about 10 to 20 or 12 to 18 amino acids in length, preferably 12, 15, or 18 amino acids in length), small organic molecule, polysaccharide, lipid, fatty acid, polynucleotide, RNAi, oligonucleotide, etc. The test compound can be in the form of a library of test compounds, such as a combinatorial or randomized library that provides a sufficient range of diversity. Test compounds are optionally linked to a fusion partner, e.g., targeting compounds, rescue compounds, dimerization compounds, stabilizing compounds, addressable compounds, and other functional moieties. Conventionally, new chemical entities with useful properties are generated by identifying a test compound (called a “lead compound”) with some desirable property or activity, e.g., inhibiting activity, creating variants of the lead compound, and evaluating the property and activity of those variant compounds. Often, high throughput screening (HTS) methods are employed for such an analysis.

A “small organic molecule” refers to an organic molecule, either naturally occurring or synthetic, that has a molecular weight of more than about 50 Daltons and less than about 2500 Daltons, preferably less than about 2000 Daltons, preferably between about 100 to about 1000 Daltons, more preferably between about 200 to about 500 Daltons.

The term “patient drug selection” refers to the selection of a drug most likely to bring about a positive result or least likely to bring about a negative result or a combination of the above.

The term “symptom” refers to any phenotypic characteristic. In some cases contemplated herein, a symptom may be detrimental to a patient having said symptom. In some cases contemplated herein, a symptom may be addressed pharmaceutically, for example to ameliorate its detrimental effects, to eliminate its detrimental effects, or to counteract its detrimental effects on the patient having said symptom. In some cases a drug to address a symptom may be known and may be regularly prescribed to a patient having said symptom.

The term “efficacy” may refer to the success that a drug may have at addressing a symptom, for example to ameliorate its detrimental effects, to eliminate its detrimental effects, or to counteract its detrimental effects on the patient having said symptom. As contemplated herein, efficacy may be reduced if an individual recipient of a drug is resistant to the effects of said drug, or if an individual recipient suffers negative side effects from administration of said drug. Efficacy for a given drug may vary among patients, and in some instances said variation may correspond to a state at one or more loci within a patient's genome. In some instances, said efficacy may be predicted in part or wholly in response to the evaluation of a patient's genetic loci. In some embodiments an efficacy may be classified into four categories, such as ‘preferential use’,′ use as directed,′ significant limitations,′ or ‘serious adverse events.’ In some embodiments efficacy evaluations may be subject to a medical doctor's review.

There are many ways to treat pain. Treatment varies depending on the cause of pain. The main treatment options are as follows:

Acetaminophen: Tylenol (Acetaminophen) is used to treat pain. Unlike several other medications for pain, Tylenol does not have anti-inflammatory effects. Often, however, in cases of chronic pain, no inflammation is at the site of the pain, and thus Tylenol may be an appropriate treatment choice. Tylenol is safe when used appropriately, but can be dangerous when used excessively. Also, Tylenol may cause unwanted effects when used with certain other medicaments.

Non-Steroidal Anti-Inflammatory Medications (NSAIDs): The NSAIDs (such as Ibuprofen, Motrin, Aleve, etc.) are most beneficial in cases of acute pain, or flare-ups in patients with chronic pain. NSAIDs are also excellent at treating inflammatory conditions including tendonitis, bursitis, and arthritis. In general, NSAID use is limited for patients with chronic pain because of concerns about the development to stomach problems. While the newer, so-called COX-2 inhibitors, such as Celebrex (celecoxib), were designed to avoid this complication, caution should still be used when using these medications for long periods of time.

Corticosteroids: As with NSAIDs, corticosteroids are powerful anti-inflammatory medications, and best used for acute pain or for flare-ups of a chronic inflammatory problem. Corticosteroids can either be taken orally (such as Medrol. Prednisone), or injected into the soft tissues or joints (cortisone injections).

Narcotics: Narcotics should be considered if pain cannot be otherwise controlled. Many narcotics can be dangerous and addicting. While narcotic medications are useful for acute pain, they also have significant side effects. The short-acting types of these medications can lead to overuse and the development of tolerance. Long-acting options have fewer side effects, and better control of chronic pain. Narcotics can become addictive when they are used for lengthy times without gradual reduction in the dose, or if the medications are taken for reasons other than pain.

Anti-Convulsants: Anti-convulsant medications are the category of medications that work to relieve nerve pain. These medications alter the function of the nerve and the signals that are sent to the brain. The most commonly prescribed anticonvulsant medication for nerve pain is called Neurontin (Gabapentin). Another option that has more recently emerged, specifically for the treatment of fibromyalgia, is called Lyrica (Pregabalin).

Local Anesthetics: Local anesthetics can provide temporary pain relief to an area. When used in the setting of chronic pain, local anesthetics are often applied as a topical patch to the area of pain. Lidoderm comes in a patch that is applied to the skin and decreases the sensitivity of this area.

The types of “analgesic drugs” are described as follows.

Narcotic analgesics include opiates, opiate derivatives, opioids, and their pharmaceutically acceptable salts. Specific examples of narcotic analgesics include alfentanil, alphaprodine, anileridine, bezitramide, buprenorphine, butorphanol, codeine, dezocine, dihydrocodeine, diphenoxylate, ethylmorphine, fentanyl, heroin, hydrocodone, hydromorphone, isomethadone, levomethorphan, levorphanol, meptazinol, metazocine, metopon, morphine, nalbuphine, nalmefene, opium extracts, opium fluid extracts, pentazocine, propoxyphene, powdered opium, granulated opium, raw opium, tincture of opium, oxycodone, oxymorphone, pethidine(meperidine), phenazocine, piminodine, racemic methadone, racemethorphan, racemorphan, sufentanil, thebaine, tramadol, and pharmaceutically acceptable salts thereof. For a detailed discussion of these and other narcotic analgesics, reference may be made to Jaffe et al., “Opioid Analgesics and Antagonists,” Goodman and Gilman's Pharmacological Basis of Therapeutics, Goodman et al., eds. 9th eds., MacMillan and Company, New York pp. S21-SS6 (1996)(“Jaffe”), which is hereby incorporated by reference.

Other narcotic analgesics and/or addictive substances that can be utilized herein include acetorphine, acetyldihydrocodeine, acetylmethadol, allylprodine, alphracetylmethadol, alphameprodine, alphamethadol, benzethidine, benzylmorphine, betacetylmethadol, betameprodine, betamethadol, betaprodine, clonitazene, cocaine, codeine methylbromide, codeine-N-oxide, cyprenorphine, desomorphine, dextromoramide, diampromide, diethylthiambutene, dihydromorphine, dimenoxadol, dimepheptanol, dimethylthiamubutene, dioxaphetyl butyrate, dipipanone, drotebanol, ethanol, ethylmethylthiambutene, etonitazene, etorphine, etoxeridine, furethidine, hydromorphinol, hydroxypethidine, ketobemidone, levomoramide, levophenacylmorphan, methyldesorphine, methyldihydromorphine, morpheridine, morphine methylbromide, morphine methylsulfonate, morphine-N-oxide, myrophin, nicocodeine, nicomorphine, nicotine, noracymethadol, norlevorphanol, normethadone, normorphine, norpipanone, phenadoxone, phenampromide, phenomorphan, phenoperidine, piritramide, pholcodine, proheptazoine, properidine, propiram, racemoramide, thebacon, trimeperidine and the pharmaceutically acceptable salts thereof.

Still other substances that can be utilized in the practice of the invention include the sedatives and hypnotics, e.g., benzodiazepines such as chlordiazepoxide, clorazepate, diazepam, flurazepam, halazepam, ketazolam, borazepam, oxazepam, prazepam, temazepam, triazolam and the pharmaceutically acceptable salts thereof, barbiturates such as amobarbital, amobarbital, barbital, butabarbital, mephobarbital, methohexital, pentobarbital, phenobarbital, secobarbital, talbutal, thiamylal and thiopental and the pharmaceutically acceptable salts thereof and other sedatives and hypnotics such as chloral hydrate, meprobamate, methaqualone, methyprylon and the pharmaceutically acceptable salts thereof.

Still other analgesics and adjuvant analgesics include (1) local anesthetics including bupivacaine, lidocaine, mepivacaine, mexiletine, tocainide and others listed in “Local Anesthetics,” Goodman and Gilman's Pharmacological Basis of Therapeutics, Goodman et al., eds. 9th eds., MacMillan and Company, New York pp. 331-347 (1996), which is hereby incorporated by reference; (2) Acetaminophen, salicylates including acetylsalicylic acid, nonsteroidal antiinflammatory drugs including propionic acid derivatives (ibuprofen, naproxen, etc), acetic acid derivatives (indomethacin, ketorolac and others), enolic acids (piroxicam and others) and cyclooxygenase II inhibitors (eg. SC-58635) and others listed in “Analgesic-antipyretic and Antiinflammatory Agents and Drugs Employed in the Treatment of Gout” Goodman and Gilman's Pharmacological Basis of Therapeutics, Goodman et al., eds. 9th eds., MacMillan and Company, New York pp. 617-657 (1996), which is hereby incorporated by reference; (3) adjuvant analgesics are used to enhance the analgesic efficacy of other analgesics (eg. opioids), to treat concurrent symptoms that exacerbate pain and provide analgesia for specific types of pain (e.g. neuropathic pain). They include corticosteroids (dexamethasone), anticonvulsants (phenytoin, carbamazepine, valproate, clonazepam and gabapentin), neuroleptics (methotrimeprazine), antidepressants (amitripline, doxepin, imipramine, trazodone), antihistamines (hydroxyzine), muscle relaxants (methocarbamol, carisoprodol, chlorzoxazone, cyclobenzaprine, gabapentin, metaxalone, baclofen, clonidine, tizanidineand other imidazoline compounds, hydantoin, dantrolene, and orphenadrine), antifolates (methotrexate) and psychostimulants (dextroamphetamine and methylphenidate) (Jacox A, et al. “Management of Cancer Pain. Clinical Practice Guideline No. 9”, AHCPR Publication No. 94-0592. Rockville, Md. Agency for Health Care Policy and Research, U.S. Department of Health and Human Services, Public Health Service, pp 65-68 (1994), which is hereby incorporated by reference).

All of the above mentioned treatment options have drawbacks, side effects, or use is limited to certain types of pain. Hence, there is still a high unmet medical need for the treatment of pain.

The term “computer-readable medium” is used herein to include any medium which is capable of storing or encoding a sequence of instructions for performing the methods described herein and can include, but not limited to, optical and/or magnetic storage devices and/or disks, and carrier wave signals.

The computer system as used here is any conventional system including a processor, a main memory and a static memory, which are coupled by bus. The computer system can further include a video display unit (e.g., a liquid crystal display (LCD) or cathode ray tube (CRT)) on which a user interface can be displayed). The computer system can also include an alpha-numeric input device (e.g., a keyboard), a cursor control device (e.g., a mouse), a disk drive unit, a signal generation device (e.g., a speaker) and a network interface device medium. The disk drive unit includes a computer-readable medium on which software can be stored. The software can also reside, completely or partially, within the main memory and/or within the processor. The software can also be transmitted or received via the network interface device.

The terms “genetic variation” or “genetic variant”, as they are used in the present description include mutations, polymorphisms and allelic variants. A variation or genetic variant is found amongst individuals within the population and amongst populations within the species.

The term “polymorphism” refers to a variation in the sequence of nucleotides of nucleic acid where every possible sequence is present in a proportion of equal to or greater than 1% of a population. A portion of a gene of which there are at least two different forms, i.e., two different nucleotide sequences, is referred to as a “polymorphic region of a gene”. A polymorphic region can be a single nucleotide, the identity of which differs in different alleles; in a particular case, when the said variation occurs in just one nucleotide (A, C, T or G) it is called a single nucleotide polymorphism (SNP).

A “polymorphic gene” refers to a gene having at least one polymorphic region.

The term “genetic mutation” refers to a variation in the sequence of nucleotides in a nucleic acid where every possible sequence is present in less than 1% of a population.

The terms “allelic variant” or “allele” are used without distinction in the present description and refer to a polymorphism that appears in the same locus in the same population.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

The term “genotype” refers to the specific allelic composition of an entire cell or a certain gene, whereas the term “phenotype’ refers to the detectable outward manifestations of a specific genotype.

As used herein, “genotyping” a subject (or DNA sample) for a polymorphic allele of a gene (s) refers to detecting which allelic or polymorphic form (s) of the gene (s) are present in a subject (or a sample). As is well known in the art, an individual may be heterozygous or homozygous for a particular allele. More than two allelic forms may exist, thus there may be more than three possible genotypes.

As used herein, the term “gene” or “recombinant gene” refers to a nucleic acid molecule comprising an open reading frame and including at least one exon and (optionally) an intron sequence. The term “intron” refers to a DNA sequence present in a given gene which is spliced out during mRNA maturation.

As used herein, the term “haplotype” refers to a group of closely linked alleles that are inherited together.

The expression “amplification” or “amplify” includes methods such as PCR, ligation amplification (or ligase chain reaction, LCR) and amplification methods. These methods are known and widely practiced in the art. See, e.g., U.S. Pat. Nos. 4,683,195 and 4,683,202 and Innis et al., 1990 (for PCR); and Wu et al. (1989) Genomics 4:560-569 (for LCR). In general, the PCR procedure describes a method of gene amplification which is comprised of (i) sequence-specific hybridization of primers to specific genes within a DNA sample (or library), (ii) subsequent amplification involving multiple rounds of annealing, elongation, and denaturation using a DNA polymerase, and (iii) screening the PCR products for a band of the correct size. The primers used are oligonucleotides of sufficient length and appropriate sequence to provide initiation of polymerization, i.e. each primer is specifically designed to be complementary to each strand of the genomic locus to be amplified.

Reagents and hardware for conducting PCR are commercially available. Primers useful to amplify sequences from a particular gene region are preferably complementary to, and hybridize specifically to sequences in the target region or in its flanking regions. Nucleic acid sequences generated by amplification may be sequenced directly. Alternatively the amplified sequence(s) may be cloned prior to sequence analysis. A method for the direct cloning and sequence analysis of enzymatically amplified genomic segments is known in the art.

Primers are designed to be the reverse-complement of the region to which they will anneal. In some embodiments primers are designed to anneal to a region flanking a DNA region to be amplified, such that the 3′OH of each primer is oriented along the genomic sequence directed toward the annealing site of the complementary primer binding site.

Primer design, synthesis and the use of primers in a nucleic acid amplification reaction such as a polymerase chain reaction are well known to one of skill in the art. A number of techniques for primer design and nucleic acid amplification are known to one of skill in the art or one familiar with molecular biology techniques generally. Primer selection, synthesis, and use in PCR reactions is reviewed in, for example, Mohini Joshi, and J. D. Deshpande, “POLYMERASE CHAIN REACTION: METHODS, PRINCIPLES AND APPLICATION” International Journal of Biomedical Research 2011 2(1):81-97, the contents of which are hereby incorporated by reference in their entirety.

In many embodiments, the disclosure herein is not limited by a single primer, primer pair, method of primer synthesis or method of nucle3ic acid amplification, such that any method of primer selection, synthesis, and use in amplification of target DNA may be suitable for use with the methods and systems disclosed herein.

“Biological sample” or “sample” refers to the biological sample that contains nucleic acid taken from a fluid or tissue, secretion, cell or cell line derived from the human body. For example, samples may be taken from blood, including serum, lymphocytes, lymphoblastoid cells, fibroblasts, platelets, mononuclear cells or other blood cells, from saliva, liver, kidney, pancreas or heart, urine or from any other tissue, fluid, cell or cell line derived from the human body. For example, a suitable sample may be a sample of cells from the buccal cavity.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present invention.

The term “a homolog of a nucleic acid” refers to a nucleic acid having a nucleotide sequence having a certain degree of homology with the nucleotide sequence of the nucleic acid or complement thereof. A homolog of a double stranded nucleic acid is intended to include nucleic acids having a nucleotide sequence that has a certain degree of homology with or with the complement thereof. In one aspect, homologs of nucleic acids are capable of hybridizing to the nucleic acid or complement thereof.

The term “interact” as used herein is meant to include detectable interactions between molecules, such as can be detected using, for example, a hybridization assay. The term interact is also meant to include “binding” interactions between molecules. Interactions may be, for example, protein-protein, protein-nucleic acid, protein-small molecule or small molecule-nucleic acid in nature.

The term “isolated” as used herein with respect to nucleic acids, such as DNA or RNA, refers to molecules separated from other DNAs or RNAs, respectively, which are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments that are not naturally occurring as fragments and would not be found in the natural state. The term “isolated” is also used herein to refer to polypeptides that are isolated from other cellular proteins and is meant to encompass both purified and recombinant polypeptides.

The term “mismatches” refers to hybridized nucleic acid duplexes that are not 100% homologous. The lack of total homology may be due to deletions, insertions, inversions, substitutions or frameshift mutations.

As used herein, the term “nucleic acid” refers to polynucleotides such as deoxyribonucleic acid (DNA), and, where appropriate, ribonucleic acid (RNA). The term should also be understood to include, as equivalents, derivatives, variants and analogs of either RNA or DNA made from nucleotide analogs, and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or RNA, the terms “adenosine”, “cytidine”, “guanosine”, and “thymidine” are used. It is understood that if the nucleic acid is RNA, a nucleotide having a uracil base is uridine.

The terms “oligonucleotide” or “polynucleotide”, or “portion,” or “segment” thereof refer to a stretch of polynucleotide residues which is long enough to use in PCR or various hybridization procedures to identify or amplify identical or related parts of mRNA or DNA molecules. The polynucleotide compositions of this invention include RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of the molecule.

As used herein, the term “label” intends a directly or indirectly detectable compound or composition that is conjugated directly or indirectly to the composition to be detected, e.g., polynucleotide so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6 ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Iain Johnson and Michelle T. Z. Spence. (Molecular Probes Handbook, A Guide to Flourescent Probes and Labeling Technologies (Invitrogen Corp; 11th ed.). (2010).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succi nimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

When a genetic marker or polymorphism “is used as a basis” for selecting a patient for a treatment described herein, the genetic marker or polymorphism is measured before and/or during treatment, and the values obtained are used by a clinician in assessing any of the following: (a) probable or likely suitability of an individual to initially receive treatment(s); (b) probable or likely unsuitability of an individual to initially receive treatment(s); (c) responsiveness to treatment; (d) probable or likely suitability of an individual to continue to receive treatment(s); (e) probable or likely unsuitability of an individual to continue to receive treatment(s); (f) adjusting dosage; (g) predicting likelihood of clinical benefits. As would be well understood by one in the art, measurement of the genetic marker or polymorphism in a clinical setting is a clear indication that this parameter was used as a basis for initiating, continuing, adjusting and/or ceasing administration of the treatments described herein.

The term “treating” as used herein is intended to encompass curing as well as ameliorating at least one symptom of the condition or disease.

A “response” implies any kind of improvement or positive response either clinical or non-clinical such as, but not limited to, measurable evidence of diminishing disease or disease progression, complete response, partial response, stable disease, increase or elongation of progression free survival, increase or elongation of overall survival, or reduction in toxicity or side effect vulnerability.

The term “likely to respond” shall mean that the patient is more likely than not to exhibit at least one of the described treatment parameters, identified above, as compared to similarly situated patients. Any drugs that are used for treatment can be used as prescribed, directed or indicated. Certain drugs may show greater efficacy or reduced side effects with certain individuals based on their genetic profile, and thus may be preferred, or alternatively, show reduced efficacy or greater side effects, or have other limitations which may then be prescribed with precaution, certain limitations or removed from use.

As used herein, the terms “increased”, “higher”, “greater”, “faster” or similar terms in association with the ability of an individual with a certain genotype to respond to a treatment shall refer to or mean having average or above average activity (the activity associated with such terms, not meant to be positive or negative) to such treatments, (e.g., faster metabolism, increased efficacy or apposingly, increased vulnerability to side effects, or increased tolerance to treatments) in comparison to similarly situated individuals with genotype(s). Alternatively, the terms “decreased”, “lower”, “reduced” or similar terms in association with the ability of individuals with a certain genotype to respond to a treatment shall mean having less or reduced response to such treatments, increased vulnerability to side effects, or reduced tolerance to treatment in comparison to similarly situated individuals with different genotype(s).

The term “preferential use” is used herein describes the use prescription or over the counter medication or drug prescribed by a physician based the genomic information received from or about the patient. The medication or drug is likely to have better than average therapeutic benefits and/or lower-than-average adverse effect risk when used in the patient with a known genotype.

The term “use as directed” is used herein describes use of a prescription or over the counter medication, drug or other product as instructed by a physician or labeling instructions for the medication used in the patient with a known genotype.

The term “may have significant limitations” is used herein describes a medication, drug or other product that is likely to have lower than average therapeutic benefits and/or higher that average adverse effect risk adverse when used in the patient with a known genotype.

The term “may cause serious adverse effects” is used herein describes any untoward medical occurrence in a patient or clinical investigation subject administered a pharmaceutical product and which does not necessarily have to have a causal relationship with this treatment. An adverse effect can also be described as a side effect. Adverse side effects can include but are not limited to hepatoxicity, cardiovascular effects, bone marrow toxicity, pulmonary toxicity, renal toxicity, central nervous system toxicity immunogenicity, hypersensitivity or death. Close monitoring or alternative medications are strongly recommended.

### General Embodiments of the Invention

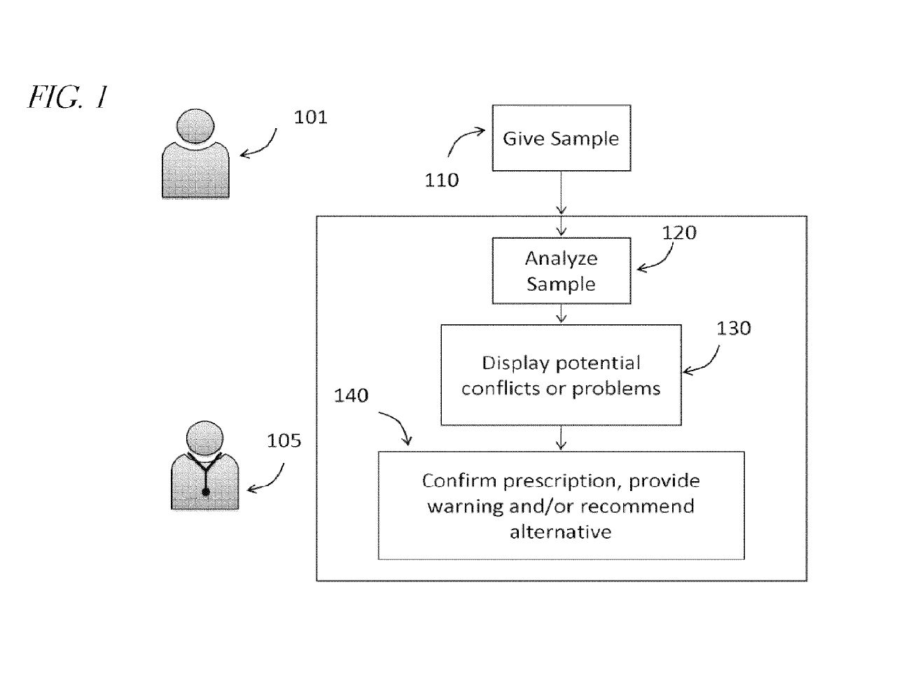

In one embodiment, as illustrated in FIG. 1, the present invention relates to systems and methods for predicting an individual's 101 likely response to a pain medication comprising genotyping genetic variations in an individual 101 to determine the individual's 101 propensity for 1) metabolizing a pain medication and 2) likely response to a medication a, and preferably 3) adverse reaction to a medication. In particular, the invention comprises analyzing 120 a biological sample 110 provided by an individual 101, typically a patient or an individual 101 diagnosed with a particular disorder, determining the individual's likely response to a particular treatment, more specifically a pain medication, and thereafter displaying 130, or further, recommending 140 a plan of action or inaction. In particular, the present invention provides a grading method and system to profile an individual's response to one or more pain medication. In an alternate embodiment, the present invention is directed to a method and system to recommend pain medications suitable for the individual.

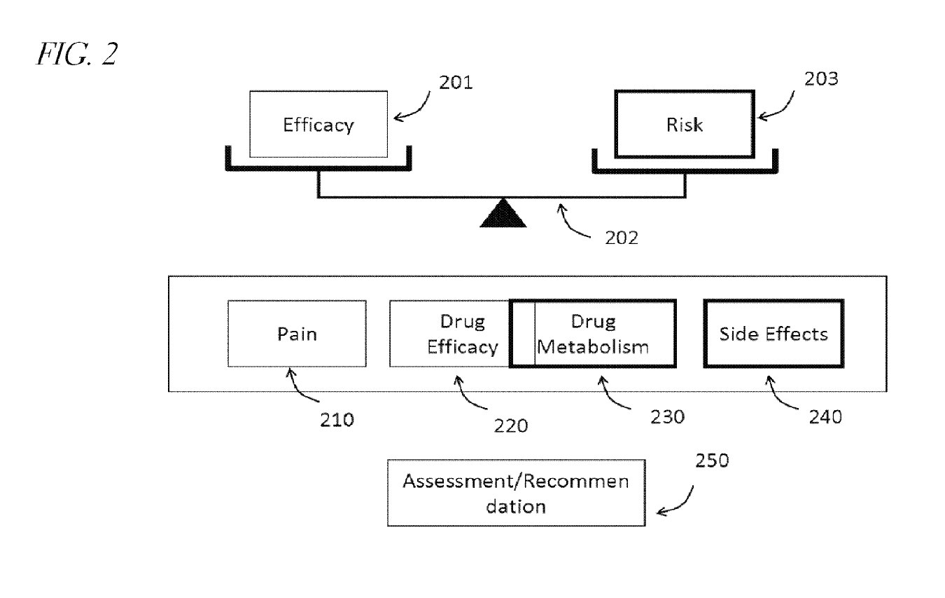

In a more preferred embodiment, as shown in FIG. 2, the present invention is directed to a method and system for analyzing an array of genetic variations related to medication or drug metabolism, drug efficacy and side effects. In a preferred method, the present invention comprises genotyping genetic variations in an individual to determine: a categorical grade to the individual's likely ability to metabolize a particular pain medication, and a categorical grade for a pain medication's potential efficacy with respect to the individual, aggregating the categorical grades, and thereafter identifying the least positive grade as the recommendation for the individual.

In a preferred embodiment, the present invention further comprises genotyping (including sequencing) an individual to determine a categorical grade to the propensity for the individual to have a negative adverse reaction to the particular pain medication.

Preferably, the individual is genotyped against a panel of at least one gene that affects the rate of drug metabolism, and a panel of genes that affect a pain medication's potential efficacy with respect to the individual. More preferably, the present invention further comprises genotyping a panel of genes that affect the propensity for the individual to have a negative adverse reaction to the particular pain medication.

As defined herein, the term “least positive” refers to the most precautionary category or measure or assessment that can be attributed to an individual based on their potential response to pain medications. For example, the assessment for an individual with respect to their response to a particular drug may be positive or normal with respect to all aspects except, for example, a potential negative adverse reaction. The potential negative reaction would be the least positive or most precautionary assessment, and would be the recommendation to the patient, e.g., the patient may be at risk for potential negative adverse reactions.

FIG. 2 can be identified as a method and system for genetically evaluating the efficacy 201 of a particular pain treatment for an individual balanced 202 against any risks 203 associated with the use of such treatment. Once a particular disorder is identified, and preferably confirmed 210, the efficacy of the drug 220 with respect to the particular individual and the disorder, is balanced against the pharmacokinetics of the medication or drug 230 and further weighted by any potential side effects 240 that the individual or the drugs may be prone to. The disorder can be assessed by genotyping the individual to determine if they are prone to such disorder or by traditional means of diagnosing such disorders. In many cases, the pharmacokinetics of the drug will affect the efficacy of the drug, e.g., tolerance or metabolism of the drug will affect the disorder and the individual, and also the side effects or any adverse effects that may arise due to the drug lingering or affecting non-desired pathways. A recommendation or assessment 250 is made based on the weighting of these factors.

In a preferred embodiment, the present invention comprises an algorithm or system, wherein a drug is assigned to categories such as one of the four categories below:

### 1. Use as Directed

### 2. Preferential Use

### 3. May Have Significant Limitations

### 4. May Cause Serious Adverse Events

For example, in one embodiment, each drug is assigned to the default category, “Use as Directed”, unless it is reassigned to another category based on genetic test result(s). In case the drug can be reassigned to multiple categories because of results from multiple genetic tests, the category that invokes most precautionary measures (e.g., least positive) will apply to the drug. For instance, a drug will be assigned to the “May Cause Serious Adverse Events” category for a patient when the patient is positive for both 1) a genotype that is associated with increased response to the drug, suggesting the “Preferential Use” category, and 2) another genotype that is associated with increased risk of serious adverse events, suggesting the “May Cause Serious Adverse Events” category.

The Input of the algorithm consists of the genotyping results of the patient.

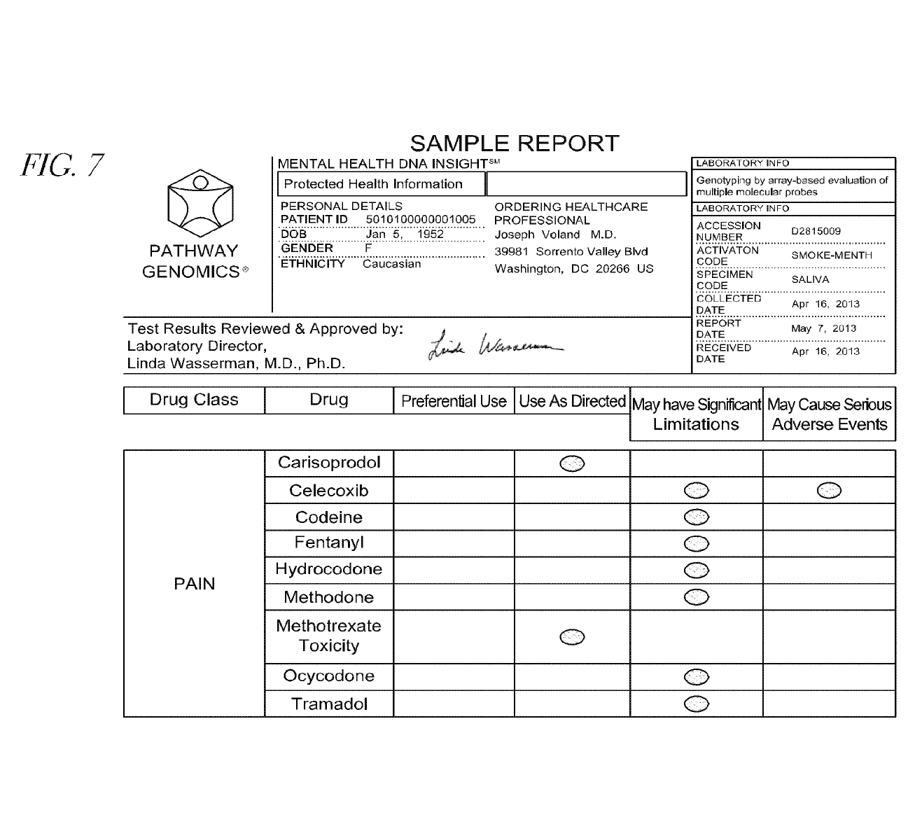

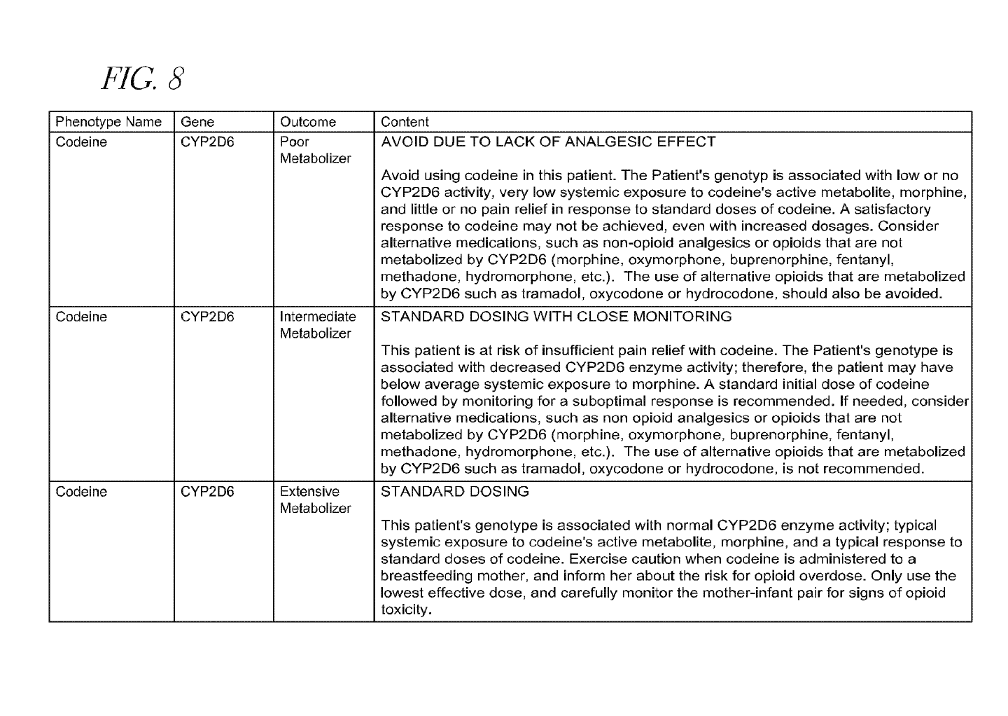

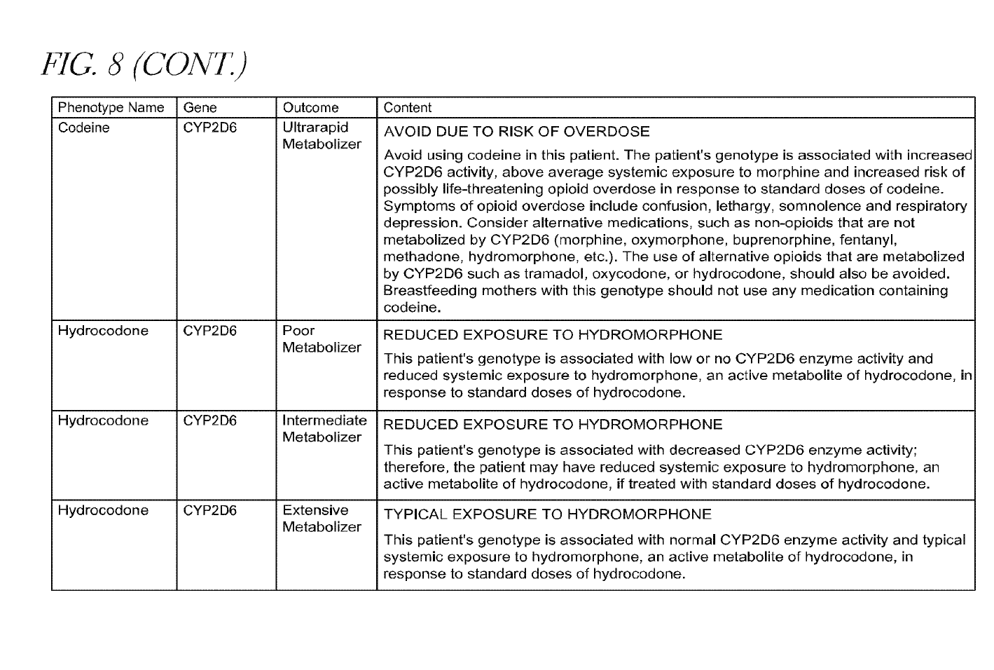

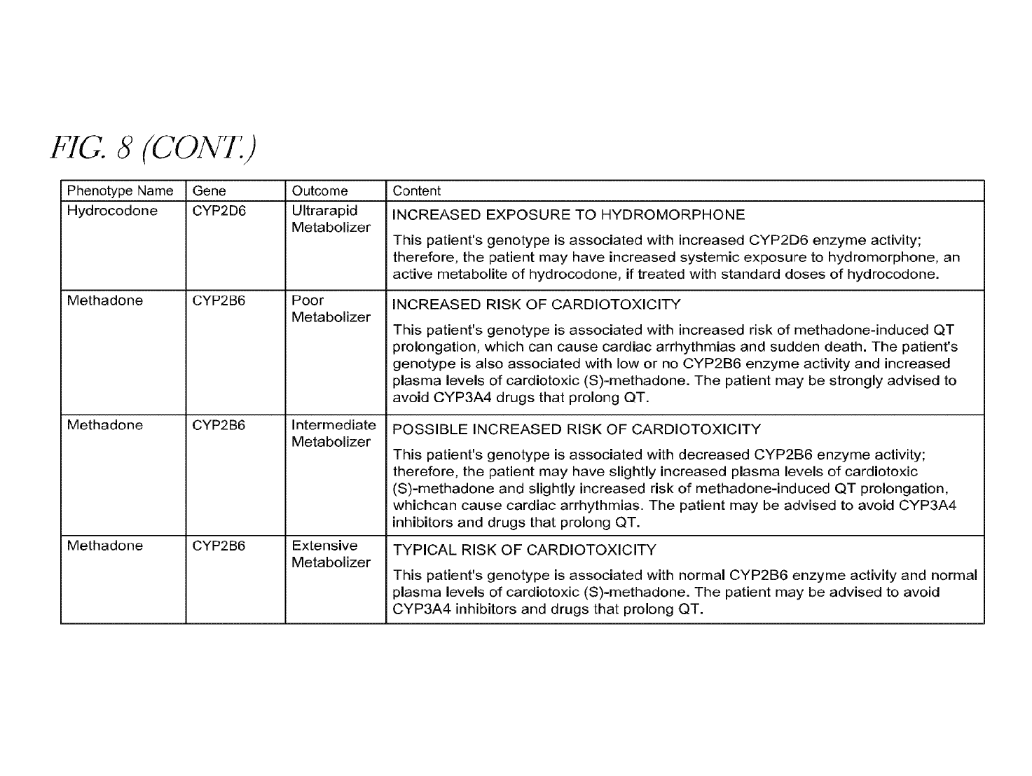

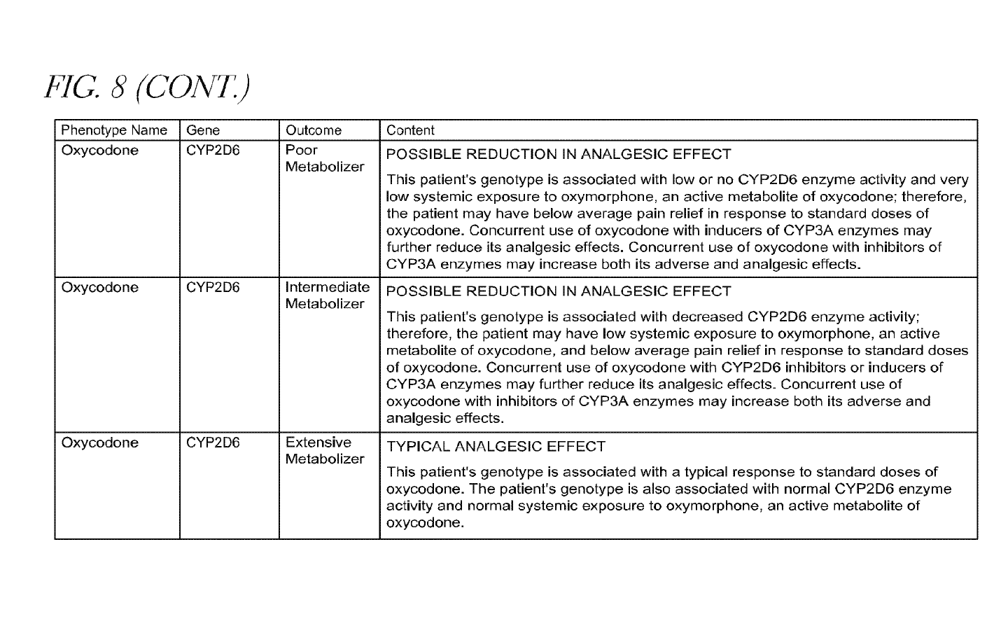

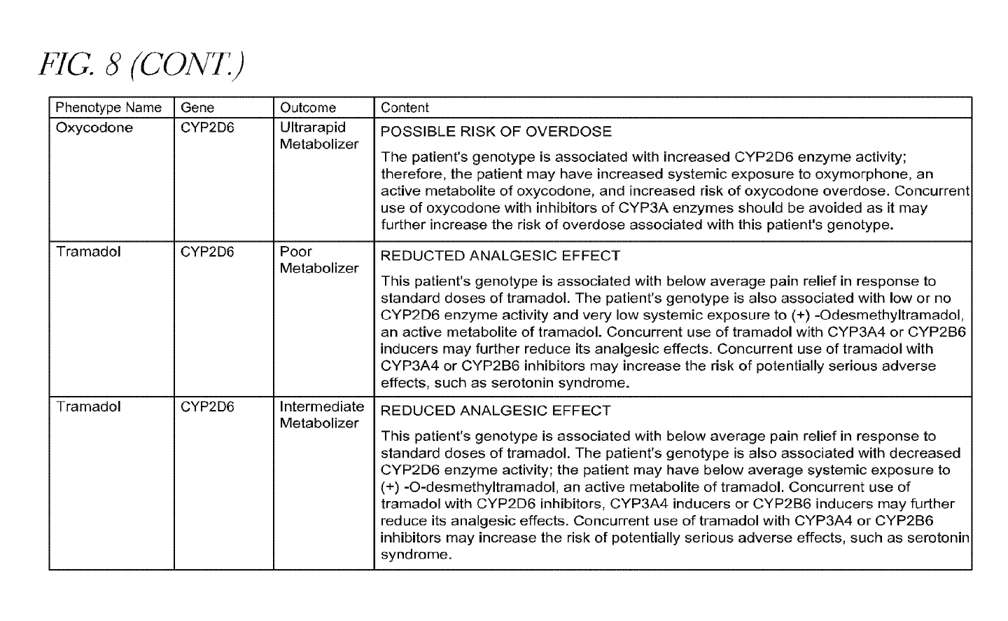

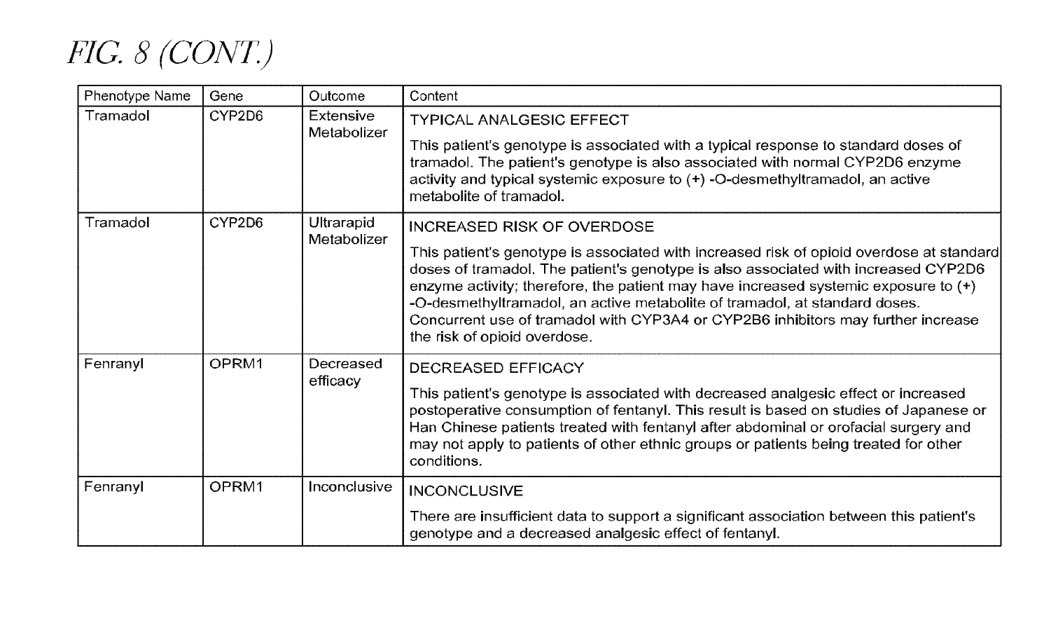

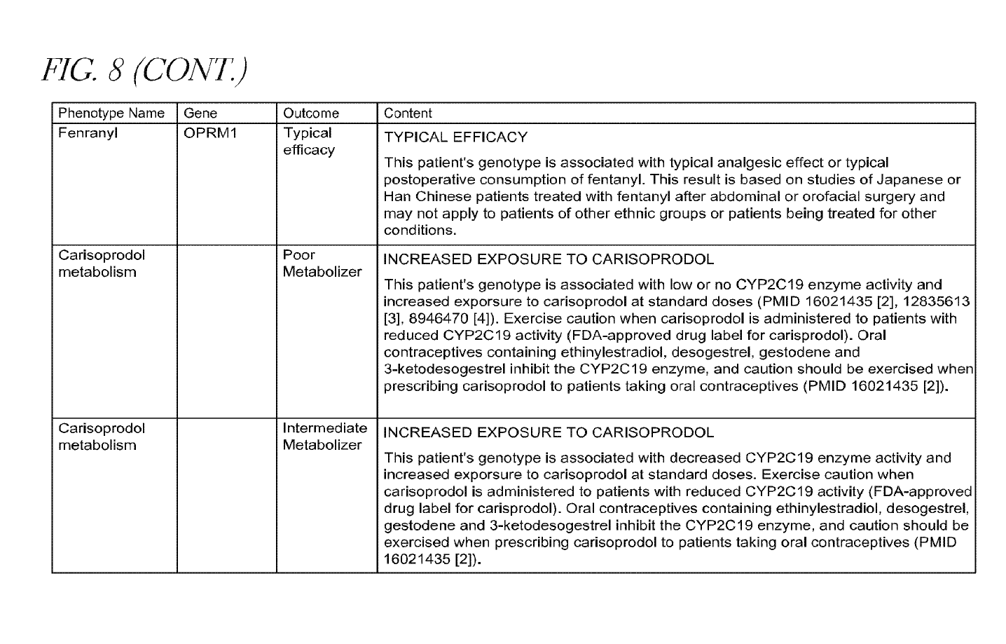

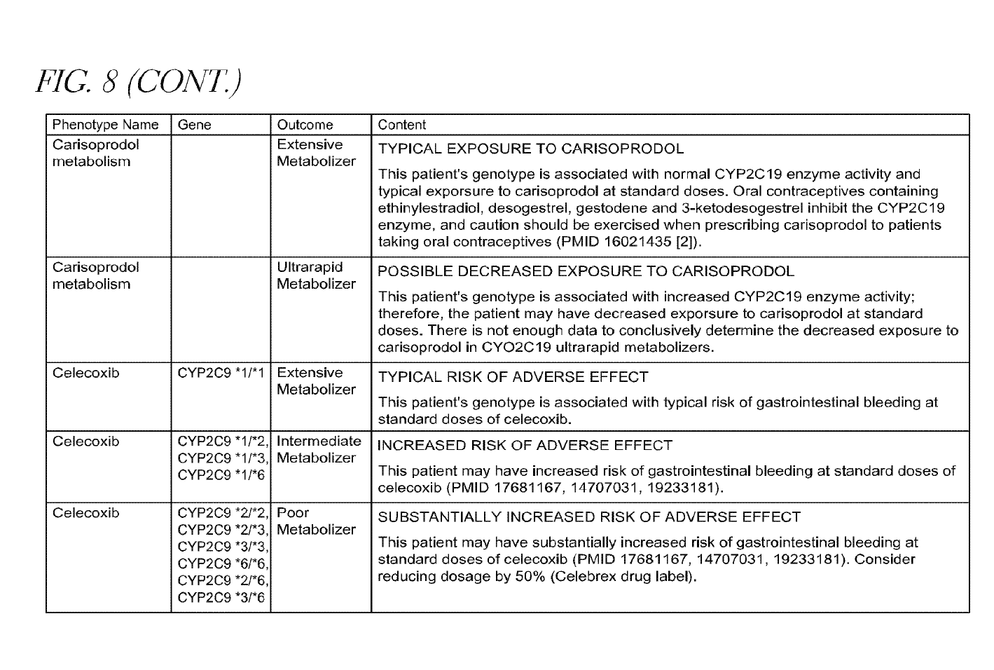

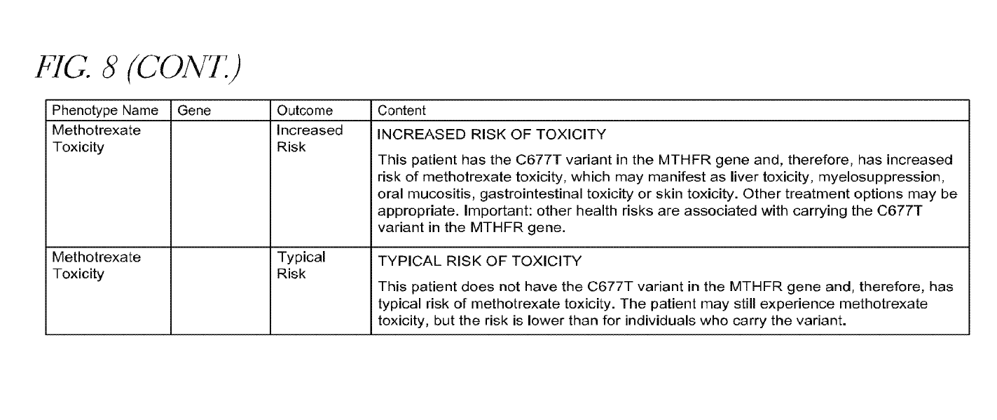

The output of the algorithm consists of the recommendation categories for all tested drugs and a text for each drug that is not assigned to the “Use as Directed” category. The text includes detailed reasons for the category assignment and, when appropriate, clinical recommendations (FIGS. 7-8).

In FIG. 8 is shown a summary of alternate information that may be included in a report such as that presented in FIG. 7. Presented are genetic loci, the specific position of the locus to be assayed, tetails of the locus, the drug for which the locus is relevant, the category of the relevance assessment, the source of the information upon which the relevancy assessment is based, and the phenotype of which the assay is related. As indicated therein, loci may be relevant to multiple drugs, categories or phenotypes. Later in FIG. 8, information is arranged by phenotype, such that the loci, outcomes, and content related to a given phenotype are readily available.

In FIG. 7 is given an example of an output report related to information of FIG. 8. FIG. 7 is not, however, a limiting example. On the contrary, any number of combinations of information of FIG. 8 may be included in a report formatted such as that of FIG. 7 but including additional or different loci, bases to assay, phenotypes, outcomes and content. Contemplated in the disclosure herein are any number of combinations of entries of FIG. 8 into reports formatted such as that in FIG. 7. That is, all combinations comprising 1, 2, 3, 4, 5, 6, 7, 8, 9, 10, 11, 12, 13, 14, 15, 16, 17, 18, 19, 20, or more than 20 entries of FIG. 8 are contemplated, to constitute one or more reports such as the report formatted in non-limiting FIG. 7.

The algorithm consists of:
A library of candidate recommendation category assignments for all drug-genotype combinations,
A library of texts for all drug-genotype combinations,
Rules for determining the final drug recommendation categories,
Rules for selecting texts for display in the test report, and
Rules for assessing the impact of incomplete test results.

In one embodiment, the present invention relates to a method of genotyping genetic variations in an individual, which is sufficiently sensitive, specific and reproducible as to allow its use in a clinical setting. The inventors have developed unique methodology with specifically designed primers and probes for use in the method.

Thus in one aspect, the invention comprises an in vitro method for genotyping genetic variations in an individual. The in vitro, extracorporeal method is for simultaneous sensitive, specific and reproducible genotyping of multiple human genetic variations present in one or more genes of a subject. The method of the invention allows identification of nucleotide changes, such as, insertions, duplications and deletions and the determination of the genotype of a subject for a given genetic variation.

A given gene may comprise one or more genetic variations. Thus the present methods may be used for genotyping of one or more genetic variations in one or more genes.

Thus a genetic variation may comprise a deletion, substitution or insertion of one or more nucleotides. In one aspect the genetic variations to be genotyped according to the present methods comprise SNPs.

Typically the individual is a human.

The invention further provides methods for detecting the single nucleotide polymorphism in the gene of interest. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

The efficacy of a drug is a function of both pharmacodynamic effects and pharmacokinetic effects, or bioavailability. In the present invention, patient variability in drug safety, tolerability and efficacy are discussed in terms of the genetic determinants of patient variation in drug pharmacokinetics (e.g., absorption, distribution, metabolism, and excretion), drug efficacy and tolerance, and propensity for adverse events. As described herein the present invention comprises testing an individual for at least one genetic variation or occurrence of genetic polymorphism in genes associated with the rate of metabolism, testing an individual for at least one genetic variation or occurrence of genetic polymorphism in genes associated with the efficacy of or tolerance to a particular pain medication, and testing an individual for at least one genetic variation or occurrence of genetic polymorphism in genes associated or related to any adverse reaction to a particular pain medication. In a preferred method, an individual is also tested to detect any genetic variation or occurrence of genetic polymorphism in genes associated with a particular indication, disease or disorder to confirm the diagnosis. Accordingly, in a more preferred embodiment, the method comprises genotyping, in parallel/sequence or independently, genetic variations in the individual to determine the risk for a particular indication, disease or disorder an individual may carry. Such genes (and polymorphisms) associated with the above are listed herein. Additional exemplary information is provided in the appendices of the present application of exemplary genetic markers that may put patients at risk for particular types of pain medications.

Listed below are genes that are associated with metabolism, efficacy, adverse reactions and risk. This list is not exhaustive, but representative of possible genes for analysis.

### Metabolism

Individual variation of drug effects in humans can be attributed to many factors. Among the factors, the rate of drug metabolism has been regarded as one of the most important ones. Drug metabolism also known as xenobiotic metabolism is used herein to refer to the biochemical modification of pharmaceutical substances or xenobiotics respectively by living organisms, usually through specialized enzymatic systems. Drug metabolism often converts lipophilic chemical compounds into more readily excreted hydrophilic products. The rate of metabolism determines the duration and intensity of a drug's pharmacological action. A genetic defect of enzymes involved in drug metabolism, particularly cytochrome P450 (CYP), has been believed to be one of the important causal factors of adverse drug reactions. The activity of the enzymes is diverse in individuals, and the enzymes are classified into PM (poor metabolizers) IM (intermediate metabolizers) EM (extensive metabolizers) and UM (ultrarapid metabolizers) depending on the degree of activity. Partly, the genetic polymorphism of the genes causes diverse activities of the enzymes.

There are multiple gene mutations for CYP causing the poor metabolizer phenotype. The occurrence of genetic polymorphism has been seen in genes for CYP1A1, CYP1A2 CYP2A6, CYP2C9, CYP2C19, CYP2D6, CYP2E1 and CYP3A5. Others implicated in drug metabolism may include: CYP1B1, CYP2B6, CYP2C8, CYP2C18, CYP3A4, UGT1A1, UGT1A4, UGT1A9, UGT2B4, UGT2B7, NAT1, NAT2, EPHX1, MTHFR, ABCB1, FM03, TPMT, and dihydropyrimidine dehydrogenase (DPD). Examples of the association of alleles of the given enzymes with a metabolic phentotype can be found in the literature.

Polymorphisms of drug-metabolizing enzymes CYP2C9, CYP2C19, CYP2D6, CYP1A1, NAT2 and of P-glycoprotein (MDR-1) in a Russian population are described in Gaikovitch et al., Eur. J. Clin. Pharmacol. 59 (2003), 303-312.

This variability is in part attributable to genetic differences that result in slowed or accelerated oxidation of many pain drugs metabolized by the cytochrome P450 (CYP450) isoenzyme system in the liver. In particular, clinically relevant variants have been identified for the isoenzymes coded by the CYP2C9, CYP2C19 and CYP2D6 genes. Polymorphisms in CYP2C9 may be important in pain patients deficient for other CYP450 enzymatic activities. For example, the influence of CYP2C9 genetic polymorphisms on pharmacokinetics of celecoxib and its metabolites is described in Kirchheiner et al., Pharmacogenetics 13 (2003), 473-480. Some of the potential consequences of polymorphic drug metabolism are extended pharmacological effect, adverse drug reactions (ADRs), lack of prodrug activation, drug toxicity, increased or decreased effective dose, metabolism by alternative deleterious pathways and exacerbated drug-drug interactions. CYP450 isoenzymes are also involved in the metabolism of endogenous substrates, including neurotransmitter amines, and have been implicated in the pathophysiology of mood disorders. CYP2D6 activity has been associated with personality traits and CYP2C9 to MDD.

The CYP3A4 enzyme is the primary metabolizer of fentanyl and oxycodone, although normally a small portion of oxycodone undergoes CYP2D6 metabolism to oxymorphone. Tramadol undergoes both CYP3A4- and CYP2D6-mediated metabolism. Methadone is primarily metabolized by CYP3A4 and CYP2B6; CYP2C8, CYP2C19, CYP2D6, and CYP2C9 also contribute in varying degrees to its metabolism. The complex interplay of methadone with the CYP system, involving as many as 6 different enzymes, is accompanied by considerable interaction potential.

Exemplary polymorphisms include:

C430T, A1075C, 818delA, T1076C and C1080G of the cytochrome P450 2C9 (CYP2C9), 2613delAGA, C2850T, G3183A, C3198G, T3277C, G4042A and 4125insGTGCCCACT of the cytochrome P450 2D6 (CYP2D6),
A-163C, A-3860G, G3534A and C558A of the cytochrome P450 1A2 (CYP1A2), G636A, G681A, C680T, A1G, IVS5+2T>A, T358C, G431A and C1297T of the cytochrome P450 2C19 (CYP2C19), 11e462Val of the cytochrome P450 1A1 (CYP1A1), G14690A, C3699T, G19386A, T29753C and G6986A of the cytochrome P450 3A5 (CYP3A5),

Pharmacogenetics is a discipline that attempts to correlate specific gene variations with responses to particular drugs. Such DNA-guided pharmacotherapy would be potentially cost effective and could spare patients from unwanted side effects by matching each with the most suitable, individualized drug and dosing regimen at initiation of pharmacotherapy. There have been strategies personalizing dosing for pain drugs according to algorithms derived from studies of blood levels. Beyond pharmacogenetics, it has become apparent that therapeutic index is a necessary concept in understanding how CYP450 polymorphism may influence personalized prescription.

A 1998 meta-analysis of 39 prospective studies in US hospitals estimated that 106,000 Americans die annually from ADRs. Adverse drug events are also common (50 per 1000 person years) among ambulatory patients, particularly the elderly on multiple medications. The 38% of events classified as ‘serious’ are also the most preventable. It is now clear that virtually every pathway of drug metabolism, transport and action is susceptible to gene variation. Within the top 200 selling prescription drugs, 59% of the 27 most frequently cited in ADR studies are metabolized by at least one enzyme known to have gene variants that code for reduced or nonfunctional proteins.

In drug treatments, the high carrier prevalence of deficient CYP450 alleles has significant implications for healthcare management. Uninformed prescribing of pain medications to patients with highly compromised biochemical activity for the CYP450 isoenzymes, may expose 50% of patients to preventable severe side effects. If these patients were carriers of gene polymorphisms resulting in deficient metabolism, their risk of adverse drug effects would substantially increase. Were DNA typing to be performed after development of drug resistance or intolerance, such information could guide subsequent pharmacotherapy and assist in diagnosing drug-induced side effects. The value of DNA typing for diagnosing severe drug side effects and treatment resistance has been documented in various case reports. Optimally, DNA typing could be performed prior to drug prescription in order to optimize therapy at the outset of drug management.

While it is well known that inter-individual variation in drug metabolism is highly dependent on inherited gene polymorphisms, the debate regarding the role of genotyping in clinical practice continues. The utility of the system described herein is to provide clinically relevant indices of drug metabolism status based on combinatorial genotypes of members of the cytochrome P450 family such as CYP2C9, CYP2C19 and CYP2D6.

UDP-glucuronosyltransferase (UGT) is an enzyme which catalyzes glucuronic acid to couple with endogenous and exogenous materials in the body. The UDP-glucuronosyltransferase generates glucuronic acid coupler of materials having toxicity such as phenol, alcohol, amine and fatty acid compound, and converts such materials into hydrophilic materials to be excreted from the body via bile or urine (Parkinson A, Toxicol Pathol., 24:48-57, 1996).

The UGT is reportedly present mainly in endoplasmic reticulum or nuclear membrane of interstitial cells, and expressed in other tissues such as the kidney and skin. The UGT enzyme can be largely classified into UGT1 and UGT2 subfamilies based on similarities between primary amino acid sequences. The human UGT1A family has nine isomers (UGT1A1, and UGT1A3 to UGT1A10). Among them, five isomers (UGT1A1, UGT1A3, UGT1A4, UGT1A6 and UGT1A9) are expressed from the liver. The UGT1A gene family has different genetic polymorphism depending on people. It is known that several types of genetic polymorphism are present with respect to UGT1A1, and UGT1A3 to UGT1A10 genes (http://galien.pha.ulaval.ca/alleles/alleles.html). The polymorphism of UGT1A genes is significantly different between races. It has been confirmed that the activity of enzymes differs depending on the polymorphism, and the polymorphism is an important factor for determining sensitivity to drug treatment. UGT1A1*6 and UGT1A1*28 are related to Gilbert Syndrome (Monaghan G, Lancet, 347:578-81, 1996). Further, various functional variants which are related to various diseases have been reported. Functional variants in the UGT1A genes include—39(TA)6>(TA)7, 211G>A, 233C>T and 686C>A of a UGT1A1 gene; 31T>C, 133C>T and 140T>C of a UGT1A3 gene; 31C>T, 142T>G and 292C>T of a UGT1A4 gene; 19T>G, 541A>G and 552A>C of a UGT1A6 gene; 387T>G, 391C>A, 392G<A, 622T>C and 701T>C of a UGT1A7 gene; and—118T9>T10, 726T>G and 766G>A of a UGT1A9 gene.

Similar to the cytochrome P450 family, the 5,1O-methylenetetrahydrofolate reductase (MTHFR) is a key enzyme for intracellular folate homeostasis and metabolism. Methylfolic acid, synthesized from folate by the enzyme MTHFR, is required for multiple biochemical effects in the brain. A primary role involves the synthesis of dopamine in the brain. Folic acid deficiency results in fatigue, reduced energy and depression. Low folate blood levels are correlated with depression and polymorphisms of the MTHFR gene (e.g. rs1801133) are closely associated with risk of depression.

MTHFR irreversibly reduces 5-Methyltetrahydrofolate which is used to convert homocysteine to methionine by the enzyme methione synthetase. The C677T SNP of MTHFR (rs1801133) has been associated with increased vulnerability to several conditions and symptoms including depression.

The nucleotide 677 polymorphism in the MTHFR gene has two possibilities on each copy of chromosome 1: C or T. 677C (leading to an alanine at amino acid 222); 677T (leading to a valine substitution at amino acid 222) encodes a thermolabile enzyme with reduced activity. The degree of enzyme thermolability (assessed as residual activity after heat inactivation) is much greater in T/T individuals (18-22%) compared with C/T (56%) and C/C (66-67%).

MTHFR gene polymorphisms include polymorphisms in the 5,1O-methylenetetrahydrofolate reductase (MTHFR) gene, including MTHFR C677T and its association with common pain symptoms including fatigue and depressed mood. These symptoms are proposed to be due to hypomethylation of enzymes which breakdown dopamine through the COMT pathway. In this model, COMT is disinhibited due to low methylation status, resulting in increased dopamine breakdown.

For unipolar depression, the MTHFR C677T polymorphism has been well described and validated.

Polymorphism in the N-acetyltransferase 1 (NAT1) polyadenylation signal (NAT1*10 allele) with higher N-acetylation activity in bladder and colon tissue is described in Bell et al., Cancer Res. 55 (1995), 5226-5229. Kukongviriyapan et al. (Eur. J. Clin. Pharmacol. 59 (2003), 277-281) describe polymorphism of N-acetyltransferase 1 and correlation between genotype and phenotype in a Thai population. Furthermore, Butcher et al., in Mol. Pharm. 57 (2000), 468-473, provide evidence for a substrate-dependent regulation of human arylamine N-acetyltransferase-1.

The suspected inhibitory potential of the over-the-counter (OTC) drug Ibuprofen on N-acetyltransferase 2 (NAT2) in vitro and in vivo and the possible implications for phenotyping procedures using caffeine as probe drug is described in Vrtic et al, Br. J. Clin. Pharmacol. 55 (2003), 191-198.

Nucleotide polymorphisms in the MDR1 gene encoding the P-glycoprotein, a transmembrane efflux pump that extrudes a wide variety of drugs, thereby reducing their intracellular access, and methods for quantitative determination of MDR1 mRNA are described in Oselin et al, Eur. J. Clin Invest. 33 (2003), 261-267.

The frequency of MRP1 genetic polymorphisms and their functional significance in Caucasians is described in Oselin et al., Eur. J. Clin. Pharmacol. 59 (2003), 347-350.

Another metabolic enzyme is flavin-containing monooxygenase 3 (FM03). The human flavin-containing monooxygenases catalyze the oxygenation of nucleophilic heteroatom-containing drugs, xenobiotics and endogenous materials. Evidence for six forms of the FMO gene exist but it is FMO form 3 (FM03) that is the prominent form in adult human liver that is likely to be associated with the bulk of FMO-mediated metabolism. An understanding of the substrate specificity of human FM03 is beginning to emerge and several examples of drugs and chemicals extensively metabolized by FM03 have been reported. Expression of FM03 is species- and tissue-specific, but unlike human cytochrome P450 (CYP450), mammalian FM03 does not appear to be inducible. Interindividual variation in FM03-dependent metabolism of drugs, chemicals and endogenous materials is therefore more likely to be due to genetic and not environmental effects. Certain mutations of the human FM03 gene have been associated with abnormal N-oxygenation of trimethylamine. Deficient N-oxygenation of trimethylamine results in a condition called trimethylaminuria. Some treatment strategies for this inborn error of metabolism are discussed. Other common variants of the FM03 gene including E158K, V257M and E308G have been observed. An overview is given in Cashman, Pharmacogenomics 3 (2002), 325-339. Polymorphisms of the fmo3 gene in caucasian and african-american populations are described in, for example, Lattard et al., Drug Metab. Dispos. 31 (2003), 854-860; Park et al., Pharmacogenetics 12 (2002), 77-80; Hernandez et al., Hum. Mutat. 22 (2003), 209-213; and Zeng et al., Zhonghua Yi Xue Yi Chuan Xue Za Zhi 20 (2003), 318-321.

Other genes associated with drug metabolism of pain drugs will be recognized by those of skill in the art.

### Efficacy and Tolerance

Likewise, polymorphisms in genes encoding the targets of medications (e.g. receptors) can alter the pharmacodynamics of the drug response by changing receptor sensitivity; the opioid receptor system (with MOP-r, KOP-r, and DOP-r receptors) and neuropeptides (β-endorphin [β-EP], dynorphins, and enkephalins) and interaction with dopaminergic systems. Drug receptor/effector polymorphisms and pharmacogenetics are described by Johnson and Lima in Pharmacogenetics 13 (2003), 525-534. Another review on published examples of inherited differences in drug metabolizing enzymes, drug transporters, and drug targets (for example, receptors) to illustrate the potential importance of inheritance in determining the efficacy and toxicity of medications in humans is provided by Evans, Gut 52: ii (2003), 10-18; for review see also Weinshilboum, N. Engl. J. Med. 348 (2003), 529-537 and Goldstein, N. Engl. J. Med. 348 (2003), 553-556.

Hence, the influence of polymorphic genes on drug action on the receptor or target molecule is well known. A drug can only exert its action on receptors and other target molecules if it can bind to it. In addition if the receptor is overexpressed in the diseased status of the cell then more drug molecules might be necessary to inactivate sufficient receptor molecules. There exists several examples where polymorphisms in genes encoding receptors or drug targets influence drug action.

TABLE
Influence of drugs on receptors and other target molecules

| | | |
|---|---|---|
| Target | Drug | Clinical Result |

| | |
|---|---|
| GTP cyclohydrolase (GCH1) | Partial analgesia (Tegeder I, |
| | Costigan M, Griffin R S, Abele A, |
| | Belfer I, et al. (2006) GTP |
| | cyclohydrolase and |
| | tetrahydrobiopterin regulate |
| | pain sensitivity and |
| | persistence. Nat Med 12: |
| | 1269-1277) |
| Catechol-o-methyltransferase | Increased/decreased pain |
| (COMT) | sensitivity (Diatchenko L, |
| | Slade G D, Nackley A G, |
| | Bhalang K, Sigurdsson A, et |
| | al. (2005) Genetic basis for |
| | individual variations in pain |
| | perception and the |
| | development of a chronic pain |
| | condition. Hum Mol Genet 14: |
| | 135-143; Kim H, Mittal D P, |
| | Iadarola M J, Dionne R A |
| | (2006) Genetic predictors for |
| | acute experimental cold and |
| | heat pain sensitivity in |
| | humans. J Med Genet 43: e40; |
| | Zubieta J K, Heitzeg M M, |
| | Smith Y R, Bueller J A, Xu K, |
| | et al. (2003) COMT |
| | val158met genotype affects |
| | mu-opioid neurotransmitter |
| | responses to a pain stressor. |
| | Science 299: 1240-1243; |
| | Diatchenko L, Nackley A G, |
| | Slade G D, Bhalang K, Belfer I, |
| | et al. (2006) Catechol-O- |
| | methyltransferase gene |
| | polymorphisms are associated |
| | with multiple pain-evoking |
| | stimuli. Pain 125: 216-224) |
| Opioid receptor mu1 | Decreased pain sensitivity, |
| (OPRM1) | decreased opioid analgesia |
| | (Fillingim R B, Kaplan L, |
| | Staud R, Ness T J, Glover T L, |
| | et al. (2005) The A118G |
| | single nucleotide |
| | polymorphism of the mu- |
| | opioid receptor gene |
| | (OPRM1) is associated with |
| | pressure pain sensitivity in |
| | humans. J Pain 6: 159-167; |
| | Lotsch J, Geisslinger G (2006) |
| | Relevance of frequent mu- |
| | opioid receptor |
| | polymorphisms for opioid |
| | activity in healthy volunteers. |
| | Pharmacogenomics J 6: 200-210 |
| Opioid receptor delta1 | Increased/decreased pain |
| (OPRD1) | sensitivity (Kim H, Neubert J K, |
| | San M A, Xu K, |
| | Krishnaraju R K, et al. (2004) |
| | Genetic influence on |
| | variability in human acute |
| | experimental pain sensitivity |
| | associated with gender, |
| | ethnicity and psychological |
| | temperament. Pain 109: 488-496) |
| Melanocortin 1 receptor | Partial analgesia, Increased |
| (MC1R) | analgesic response (Mogil J S, |
| | Ritchie J, Smith S B, Strasburg K, |
| | Kaplan L, et al. (2005) |
| | Melanocortin-1 receptor gene |
| | variants affect pain and mu- |
| | opioid analgesia in mice and |
| | humans. J Med Genet 42: |
| | 583-587; Mogil J S, Wilson S G, |
| | Chesler E J, Rankin A L, |
| | Nemmani K V, et al. (2003) |
| | The melanocortin-1 receptor |
| | gene mediates female-specific |
| | mechanisms of analgesia in |
| | mice and humans. Proc Natl |
| | Acad Sci USA 100: 4867-4872) |
| Transient receptor potential | Increased/decreased pain |
| A1 (TRPA1) | sensitivity (Kim H, Mittal D P, |
| | Iadarola M J, Dionne R A |
| | (2006) Genetic predictors for |
| | acute experimental cold and |
| | heat pain sensitivity in |
| | humans. J Med Genet 43: e40) |
| Transient receptor potential | Decreased pain sensitivity |
| V1 (TRPV1) | (Kim H, Neubert J K, San M A, |
| | Xu K, Krishnaraju R K, et al. |
| | (2004) Genetic influence on |
| | variability in human acute |
| | experimental pain sensitivity |
| | associated with gender, |
| | ethnicity and psychological |
| | temperament. Pain 109: 488-496; |
| | Park J J, Lee J, Kim MA, |
| | Back S K, Hong S K, et al. |
| | (2007) Induction of total |
| | insensitivity to capsaicin and |
| | hypersensitivity to garlic |
| | extract in human by decreased |
| | expression of TRPV1. |
| | Neurosci Lett 411: 87-91) |
| ATP-binding cassette, B1 | Altered morphine sensitivity |
| (ABCB1) | (Campa D, Gioia A, Tomei A, |
| | Poli P, Barale R (2007) |
| | Association of ABCB1/MDR1 |
| | and OPRM1 gene |
| | polymorphisms with morphine |
| | pain relief. Clin Pharmacol |
| | Ther 83: 559-566) |
| Fatty acid amide hydrolase | Increased pain sensitivity |
| (FAAH) | (Kim H, Mittal D P, Iadarola M J, |
| | Dionne R A (2006) |
| | Genetic predictors for acute |
| | experimental cold and heat |
| | pain sensitivity in humans. J |
| | Med Genet 43: e40) |
| Purinoreceptors (P2RX7) | Among 354 women with post- |
| | mastectomy pain, three single- |
| | nucleotide polymorphisms |
| | (SNPs) in P2RX7 were |
| | associated with pain intensity. |
| | Women with an allele known |
| | to heighten pore function |
| | tended to report more intense |
| | pain, whereas those with a |
| | low-functioning allele |
| | reported lower pain. In a |
| | separate cohort of 743 patients |
| | with osteoarthritis, one of the |
| | pore-promoting SNPs was |
| | associated with the risk of |
| | clinically relevant pain. (Sorge |
| | et al., Genetically determined |
| | P2X7 receptor pore formation |
| | regulates variability in chronic |
| | pain sensitivity. Nat Med. |
| | 2012 Mar. 25.) |

COMT genotype is highly associated with human pain perception. There are three major COMT haplotypes (low pain sensitivity (LPS), average pain sensitivity (APS) and high pain sensitivity (HPS)) that determine COMT enzymatic activity, encompassing ˜96% of the examined genotypes. As indicated by the nomenclature, the LPS haplotype is associated with low pain sensitivity, APS is associated with higher pain sensitivity, and HPS with the highest sensitivity to pain. Collectively, these three haplotypes account for about—11% of the variability in pain perception. Given the inevitably polygenic nature of pain perception, the magnitude of the effect of COMT haplotypes on pain sensitivity is substantial. Indeed, quantitative trait locus (QTL) mapping studies for related traits in mice have shown that each single QTL usually accounts for 5 to 25% of the overall variance in nociceptive sensitivity (Mogil et al. (2003); Abiola et al. (2003)).

The combination of synonymous and nonsynonymous SNPs within COMT haplotypes can produce effects on protein function that exceed the effects of individual SNPs. The presently disclosed subject matter provides evidence to show that genomic variations in the COMT gene do not alter the amount of COMT mRNA, suggesting that the differences in enzymatic activity result from differences in protein translation. The fact that expressed cDNA constructs, which differed in only three SNPs rs4633, rs4818, and rs4680 (val 158 met), showed more than an 11-fold difference in expressed enzyme activity, confirms that the observed association between haplotypes and pain sensitivity can be caused by combinations of these three SNPs and not necessarily by other SNPs in the haploblock situated in the 5′ or intronic region of the COMT gene that can affect RNA transcription. Without desiring to be limited by theory, interactions between SNPs can possibly have profound effects on the secondary mRNA structure, which controls the efficacy of protein translation. The identification of new functional haplotypes disclosed herein suggests that haplotype reconstruction can provide important insights into relationship between COMT polymorphism, human pain sensitivity, and somatosensory disorders. Furthermore, COMT inhibition in rodents results in a robust increase in pain sensitivity. The presently disclosed subject matter provides evidence that COMT activity regulates pain sensitivity and strongly suggests that the observed association between CO/WTgenotype and pain perception in humans is not epiphenomenal. The presently disclosed subject matter represents the first demonstration of an association between a genetic polymorphism that impacts pain sensitivity and the risk for myogenous temporomandibular disorder (TMD), which is a highly prevalent musculoskeletal pain condition (i.e., somatosensory disorder). The presence of even a single high COMT activity (LPS) haplotype diminishes by as much as 2.3 times, the risk of developing TMD. The risk ratio of 2.3 is of a magnitude comparable to genetic risk factors for other multifactorial conditions such as schizophrenia and is similar to other predictors of TMD, such as a history of chronic pain at other body sites. The clinical relevance of this novel finding is best quantified by the measure of population attributable risk for having HPS and/or APS, which was 29% in the particular cohort of women subjects studied in the Example presented belows, indicating that nearly one third of new TMD cases can be attributed to this COMT genotype.

### ADRB3 Genotypes

The presently disclosed subject matter also provides that common genetic variants of ADRB3, comparable to COMT and ADRB2, can also influence human psychological traits that influence pain sensitivity and the risk of developing a sensory disorder. Particularly, there are three major ADRB3 haplotypes (H1, H2, H3) that determine ADRB2 expression and activity, as well as other rare haplotypes.

By way of elaboration, the data presented herein based on the determined association analysis of ADRB3 haplotypes with pain responsiveness and somatization score, demonstrates that subjects bearing H2 or H3 haplotypes of ADRB3 can be predicted to have lower risk for developing somatosensory disorders, including TMD.

Further, with regard to predicting somatization in a subject based on genotyping of the subject with regard to ADRB3 haplotype, subjects bearing a H3 haplotype have a lower PILL somatization score than those who do not carry a H3 allele. Consistent with this observation, H1/H3 heterozygotes also have low pain responsiveness.

The mu-opioid receptor (OPRM1) is the primary binding site of action for many opioid drugs and for binding of beta-endorphins. In addition, endogenous opioid peptides, such as enkephalins, endorphins and dynorphins exert a wide spectrum of physiological and behavioural effects via the MOR, including effects on pain perception, mood, motor control and autonomic functions [Raynor K, Kong H, Chen Y, Yasuda K, Yu L, Bell G I, Reisine T. Pharmacological characterization of the cloned kappa-, delta-, and mu-opioid receptors. Mol Pharmacol. 1994; 45:330-334, Onali P, Olianas M C. Naturally occurring opioid receptor agonists stimulate adenylate cyclase activity in rat olfactory bulb. Mol Pharmacol. 1991; 39:436-441]. One of the effects of opiate and alcohol use is to increase release of beta-endorphins, which subsequently increases release of dopamine and stimulates cravings. Naltrexone is an opioid antagonist used to treat abuse of opiates, alcohol, and other substances. Naltrexone binds to OPRM1, preventing beta-endorphin binding and subsequently reducing the craving for substances of abuse.

The A355G polymorphism (rs1799971) in exon 1 of the OPRM1 gene (OPRM1) results in an amino acid change, Asn102Asp. Historically, this mutation has been referred to in the literature as 118A->G (Asn40Asp). (2) The G allele leads to loss of the putative N-glycosylation site in the extracellular receptor region, causing a decrease in OPRM1 mRNA and protein levels, but a 3-fold increase in beta-endorphin binding at the receptor. (3) Studies have shown individuals who carry at least 1 G allele have significantly better outcomes with naltrexone therapy including lower rate of relapse (P=0.044), a longer time to return to heavy drinking, and <20% relapse rate after 12 weeks of treatment compared with individuals who are homozygous for the A allele (55% relapse rate). (4) Other studies indicated that 87.1% of G allele carriers had a good clinical outcome, compared with only 54.8% of individuals with the A/A genotype (odds ratio, 5.75; confidence interval, 1.88-17.54). (1) A haplotype-based approach confirmed that the single OPRM1 355A->G locus was predictive of response to naltrexone treatment.

Frequency of the 355G allele varies with ethnicity but ranges between 10% and 40% (European 20%, Asian 40%, African American 10%, and Hispanic 25%).

Other markers contemplated by the present invention include one or more of the following genes associated with pain including: sodium channel NaV1.7 (SCN9A), PNPG5, NMDA receptor, HCN-2, F2, F5, βarrestin2, stat2, MTHFR, A2a, melanocortin-1, NMDA, NK1, 5HT3, ABCB1, ABCC2, ABRB2, 5HT2a, ILIA, IL1B, IL2, IL4, IL6, IL8, IL10, IL1 2, IL1 3, IL18, IL-IRa, PTGS1, PTGS2, STAT6, TGFβ, SCN9A, Nav1.7, P2RX4, P2RX7, TNFa, TNFβ, TRPA1, TRPV1 1 FAAH, GCHI, NOS1, GIRKe, GABA-A, and HLA-DRB1.

### Side Effects/Adverse Effect

In a large patient population, a medication that is proven efficacious in many patients often fails to work in some other patients. Furthermore, when it does work, it may cause serious side effects, even death, in a small number of patients. Adverse drug reactions are a principal cause of the low success rate of drug development programs (less than one in four compounds that enters human clinical testing is ultimately approved for use by the U.S. Food and Drug Administration (FDA)). Adverse drug reactions are generally undesired effects, e.g., side effects, that can be categorized as 1) mechanism based reactions and 2) idiosyncratic, “unpredictable” effects apparently unrelated to the primary pharmacologic action of the compound. Although some side effects appear shortly after administration, in some instances side effects appear only after a latent period. Adverse drug reactions can also be categorized into reversible and irreversible effects. The methods of this invention are useful for identifying the genetic basis of both mechanism based and ‘idiosyncratic’ toxic effects, whether reversible or not. Methods for identifying the genetic sources of interpatient variation in efficacy and mechanism based toxicity may be initially directed to analysis of genes affecting pharmacokinetic parameters, while the genetic causes of idiosyncratic adverse drug reactions are more likely to be attributable to genes affecting variation in pharmacodynamic responses or immunological responsiveness. Provided herein are a list of pharmaceutical drugs, psychiatric medications and other compounds and their possible adverse effects, significant limitations and other side effects set forth in FIG. 8.

A 1998 meta-analysis of 39 prospective studies in US hospitals estimated that 106,000 Americans die annually from ADRs. Adverse drug events are also common (50 per 1000 person years) among ambulatory patients, particularly the elderly on multiple medications. The 38% of events classified as ‘serious’ are also the most preventable. It is now clear that virtually every pathway of drug metabolism, transport and action is susceptible to gene variation. Within the top 200 selling prescription drugs, 59% of the 27 most frequently cited in ADR studies are metabolized by at least one enzyme known to have gene variants that code for reduced or nonfunctional proteins.

A number of compounds are associated with adverse effects that may manifest greater in those individuals showing certain genetic variability. In a particular aspect of the present invention, the invention comprises genotyping genes that increase or decrease for drug hypersensitivity in individuals, including TNFalpha (TNFa) gene, MICA, MICB, and/or HLA genes.

### TNFalpha

The immunologic effector molecule Tumor Necrosis Factor alpha (TNFa) is known to be polymorphic, and a number of polymorphisms have been reported in the TNFa promoter region. Some reports indicate that such promoter polymorphisms influence immunologic disease (Bouma et al., Scand. J. Immunol. 43: 456 (1996); Allen et al., Mol. Immunology 36: 1017 (1999)), whereas others suggest that observed associations between TNFa polymorphisms and disease occurrence are not due to functional effects of TNFa, but due to the linkage disequilibrium of TNFa with selectable HLA alleles (Uglialoro et al., Tissue Antigens, 52: 359 (1998)). A list of TNFa promoter polymorphisms is provided by Allen et al., Mol. Immunology 36: 1017 (1999). Due to variation in reported sequences and numbering, the G (-237) A polymorphism has also been referred to as G-238A, and the G (-308) A polymorphism is located at the -307 position on the above sequence. A further polymorphism, C (-5,100) G, investigated in the present research was an C/G polymorphism in the 5′untranslated region of TNFa.

A number of the TNFa promoter polymorphisms observed to date are G/A polymorphisms clustered in the region of -375 to -162 bp; that some of these polymorphisms lie within a common motif; and suggest that the motif could be a consensus binding site for a transcriptional regulator or might influence DNA structure. The G/A polymorphism at -237 has been reported to affect DNA curvature (D'Alfonso et al., Immunogenetics 39: 150 (1994)). Huizinga et al. (J. Neuroimmunology 72: 149, 1997) reported significantly less TNFa production by LPS-stimulated cells from individuals heterozygous (G/A) at -237 (compared to G/G individuals); however, a separate study did not observe these effects (Pociot et al., Scand. J. Immunol. 42: 501, 1995). The G (-237) A polymorphism has also been reported to affect autoimmune disease (Brinkman et al., Br. J. Rheumatol. 36: 516 1997 (rheumatoid arthritis); Huizinga et al., J. Neuroimmunology 72: 149 1997 (multiple sclerosis); Vinasco et al., Tissue Antigens, 49: 74 1997 (rheumatoid arthritis)) and infectious disease (Hohler et al., Clin. Exp. Immunol. 111: 579 1998 (hepatitis B); Hohler et al., J. Med. Virol. 54: 173 1998 (hepatitis c)).

As is well known genetics, nucleotide and amino acid sequences obtained from different sources for the same gene may vary both in the numbering scheme and in the precise sequence. Such differences may be due to inherent sequence variability within the gene and/or to sequencing errors. Accordingly, reference herein to a particular polymorphic site by number (e.g., TNFa G-238A) will be understood by those of skill in the art to include those polymorphic sites that correspond in sequence and location within the gene, even where different numbering/nomenclature schemes are used to describe them.

### HLA

The HLA complex of humans (major histocompatibility complex or MHC) is a cluster of linked genes located on chromosome 6. (The TNFa and HLA B loci are in proximity on chromosome 6). The HLA complex is classically divided into three regions: class I, II, and III regions (Klein J. In: Gotze D, ed. The Major Histocompatibility System in Man and Animals, New York: Springer-Verlag, 1976: 339-378). Class I HLAs comprise the transmembrane protein (heavy chain) an a molecule of beta-2 microglobulin. The class I transmembrane proteins are encoded by the HLA-A, HLA-B and HLA-C loci. The function of class I HLA molecules is to present antigenic peptides (including viral protein antigens) to T cells. Three isoforms of class II MHC molecules, denoted HLA-DR, -DQ, and -DP are recognized. The MHC class II molecules are heterodimers composed of an alpha chain and a beta chain; different alpha- and beta-chains are encoded by subsets of A genes and B genes, respectively. Various HLA-DR haplotypes have been recognized, and differ in the organization and number of DRB genes present on each DR haplotype; multiple DRB genes have been described. Bodmer et al., Eur. J. Immunogenetics 24: 105 (1997); Andersson, Frontiers in Bioscience 3: 739 (1998).

The MHC exhibits high polymorphism; more than 200 genotypical alleles of HLA-B have been reported. See e.g., Schreuder et al., Human Immunology 60: 1157-1181 (1999); Bodmer et al., European Journal of Immunogenetics 26: 81-116 (1999). Despite the number of alleles at the HLA-A, HLA-B and HLA-C loci, the number of haplotypes observed in populations is smaller than mathematically expected. Certain alleles tend to occur together on the same haplotype, rather than randomly segregating.

This is called linkage disequilibrium (LD) and may be quantitated by methods as are known in the art (see, e.g., Devlin and Risch, Genomics 29: 311 (1995); B S Weir, Genetic Data Analysis II, Sinauer Associates, Sunderland, Md. (1996)). “Linkage disequilibrium” refers to the tendency of specific alleles at different genomic locations to occur together more frequently than would be expected by chance.

Assessing the risk of a patient for developing an adverse drug reaction in response to a drug, can be accomplished by determining the presence of an HLA genotypes including HLA-B allele selected from the group consisting of HLA-B*1502, HLA-B*5701, HLA-B*5801 and HLA-B*4601, wherein the presence of the HLA-B allele is indicative of a risk for an adverse drug reaction. Other drugs include carbazapine, oxcarbazepine, licarbazepine, allopurinol, oxypurinol, phenytoin, sulfasalazine, amoxicillin, ibuprofen, and ketoprofen. Other subtypes of HLA-B15, B58 or B46, such as HLA-B*1503 or *1558, can also be used to predict the risk for developing an ADR.

More specifically, HLA-B*1502 being associated with carbamazepine-specific severe cutaneous reactions and other forms of hypersensitivity, HLA-B*5701 being associated with abacavir hypersensitivity, HLA-B*5801 being associated with allopurinol-induced severe cutaneous adverse reactions, HLA-A29, -B 12, -DR7 being associated with sulfonamide-SJS, HLA-A2, B 12 being associated with oxicam-SJS, HLA-B59 being associated with methazolamide-SJS, HLA-Aw33, B17/Bw58 being associated with allopurinol-drug eruption, HLA-B27 being associated with levamisole-agranulocytosis, HLA-DR4 being associated with hydralazine-SLE, HLA-DR3 being associated with penicillamine toxicity, HLA-B38, DR4, DQw3 being associated with clozapine-agranulocytosis, HLA-A24, B7, DQwI being associated with dipyrone-agranulocytosis. Preferably, the HLA genotype is selected from the group consisting of HLA-B*1502 being associated with carbamazepine-specific severe cutaneous reactions and other forms of hypersensitivity, HLA-B*5701 with abacavir hypersensitivity and HLA-B*5801 with allopurinol-induced severe cutaneous adverse reactions, and preferably being HLA-B*1502.

### MICA and MICB

The MHC (HLA) class I chain-related gene A (MICA) and MHC (HLA) class I chain-related gene B (MICB) belong to a multicopy gene family located in the major histocompatibility complex (MHC) class I region near the HLA-B gene. They are located within a linkage region on chromosome 6p around HLA-B and TNFalpha. The encoded MHC class I molecules are induced by stress factors such as infection and heat shock, and are expressed on gastrointestinal epithelium.

MICA is reported as highly polymorphic. The occurrence of MICA single nucleotide polymorphisms in various ethnic groups is reported by Powell et al., Mutation Research 432: 47 (2001). Polymorphisms in MICA have been reported to be associated with various diseases, although in some cases the association was attributable to linkage disequilibrium with HLA genes. See, e.g., Salvarani et al. J Rheumatol 28: 1867 (2001); Gonzalez et al., Hum Immunol 62: 632 (2001); Seki et al., Tissue Antigens 58: 71 (2001).

Various polymorphic forms of MICB have been reported (see, e.g., Visser et al., Tissue Antigens 51: 649 (1998); Kimura et al., Hum Immunol 59: 500 (1998); Ando et al., Immunogenetics 46: 499 (1997); Fischer et al., Eur J Immunogenet 26: 399 (1999)).

More genes affecting adverse reactions: ADRB3, ANKK1, ASTN2, ATF7IP2, BAT2, BAT3, BRUNOL4, CDH13, CERKL, CLCN6, MTHFR, CLMN, FHOD3, GNB3, GPR98, GRIA3, KIRREL3, LEP, LEPR, LOC729993, LTA, TNF, MC4R, MEIS2, NRG3, NUBPL, PALLD, PMCH, PPARD, PRKAA1, PRKAR2B, RNF144A, SCN1A, SLCO3A1, and SOX5.

| Target | Drug | Clinical Result |
|---|---|---|
| Beta-Adrenergic Receptor | Beta2-agonists (e.g. | Bronchodilatator response is |
| | Albuterol) | dependent on specific |
| | | haplotype combinations. |
| | | Agonist mediated efficacy is |
| | | dependent on polymorphisms |
| Dopamine Transporter | L-Dopa | Influence on drug induced |
| (9 × 40bpVariable Number of | | psychosis and dyskinesa |
| Tandem Repeats) | | |
| 5-Lipoxygenase (ALOX5) | Zileuton | No effect in patients who |
| | | share a specific tandem repeat |
| | | in the promoter. |
| Apolipoprotein E | Tacrine | ApoE negative Alzheimer |
| | | patients |
| MGMT (0 6_methylguanine- | Alkylating Agents | Promoter methylation results |
| DNA methyltransferase) | | in good survival prognosis for |
| | | glioma patients |
| KCNE2 (T8A in MiRP 1) | Sulfamethoxazol, | Drug induced Long-QT- |
| | Procainamid, Oxatomid | Syndrome |
| Glycoprotein IIIa^{(PLA1/A2)} | Aspirin or glycoprotein | Antiplatelet effect59 |
| subunit of glycoprotein | IIb/IIIa inhibitors (e.g., | Reduced response in patients |
| IIb/IIIa | Abciximab) | carrying the PL^{A2} |
| | | polymorphism |
| CETP (B1/B2) | Pravastatin | Slower development of |
| | | arteriosclerosis in B1B1 |
| | | patients |
| Alpha-Adducin | Hydrochlorothiazide | A polymorphism of the alpha- |
| | | subunit of adducin, Gly460-- |
| | | >Trp, may affect membrane |
| | | ion transport and be associated |
| | | with human EH (essential |
| | | hypertension). Higher |
| | | sensitivity in patients who |
| | | share the 460Gly/Trp |
| | | polymorphism |
| ACE (I/D) | ACE inhibitors (e.g., enalapril, | Better response of patients |
| | Enalaprilat) | bearing the ACE-II-Allele |
| | Fluvastatin | COMT Genotypes |
| | | Renoprotective effects, blood- |
| | | pressure reduction, reduction |
| | | in left ventricular mass, |
| | | endothelial function |
| | | Lipid changes (e.g., reductions |
| | | in low-density lipoprotein |
| | | cholesterol and apoliprotein |
| | | B); progression or regression |
| | | of coronary atherosclerosis |
| Arachidonate 5-lipoxygenase | Leukotriene inhibitors | Improvement in FEV₁ 42 |
| Bradykinin B2 receptor | ACE inhibitors | ACE-inhibitor-induced cough |
| | | 51 |
| Dopamine receptors (D2, D3, | Antipsychotics (e.g. | Antipsychotic response (D2, |
| D4) | haloperidol, clozapine) | D3, D4), antipsychoticinduced |
| | | tardive dyskinesia (D3), |
| | | antipsychotic-induced |
| | | acute akathisia (D3) 52-56 |
| Estrogen receptor-a | Hormone-replacement therapy | Increase in bone mineral |
| Conjugated estrogens | | density57 |
| | | Increase in high-density |
| | | lipoprotein cholesterol 58 |
| Serotonin transporter | Antidepressants (e.g., | 5-Hydroxytryptamine |
| (5-hydroxytryptamine) | clomipramine, | neurotransmission, |
| | fluoxetine, paroxetine) | antidepressant |
| | | response 60-62 |

Preferably, one or more genetic variations are evaluated in each of the categories. For example, one or more mutations, polymorphisms and/or alleles are evaluated in one or more genes in each of the categories. Preferably, one or more genetic variations, e.g., polymorphisms, are evaluated in multiple genes. For example, one or more polymorphisms may be evaluated for combinations of CYP1A2, CYP2C19, CYP2D6, and/or UGT1A4. In a more preferred method, there are two or more genetic variations genotyped in a panel, and more preferably three, four, five, six, seven, eight, nine, ten, eleven, twelve, thirteen, fourteen, fifteen or more genes in a panel.

Although the genes discussed herein are listed in separate categories for convenience in the present application, such genes may be associated in other categories. For example, genetic variations listed within the risk category may affect genes within efficacy, metabolism, and/or adverse effects. Or a gene associated with metabolism of drugs may affect efficacy (e.g., neurotransmitter activity), adverse effect and/or risk. Or a gene associated with efficacy of drugs may affect metabolism, adverse effect and/or risk. Or a gene associated with adverse effect of drugs may affect efficacy (e.g., neurotransmitter activity), metabolism and/or risk. However, generally, those of skill in the art will look at the effect of the genetic variation to determine which category a particular gene will be categorized in the present invention. For example, a serotonin receptor 2A and 2C are associated with adverse reactions to paroxetine and fluvoxamine, and atypical antipsychotic-induced weight gain and thus categorized and associated with adverse reactions/side effects, although listed herein within efficacy. Serotonin receptors and transporter genes affect the efficacy of certain drugs through different mechanisms such as transport, inhibition, agonism and the like. Similarly, although listed within genes associated with metabolism, the high carrier prevalence of deficient CYP450 alleles may expose 50% of patients to preventable severe side effects. If these patients were carriers of gene polymorphisms resulting in deficient psychotropic metabolism, their risk of adverse drug effects would substantially increase. Were DNA typing to be performed after development of drug resistance or intolerance, such information could guide subsequent pharmacotherapy and assist in diagnosing drug-induced side effects. The value of DNA typing for diagnosing severe drug side effects and treatment resistance has been documented in various case reports. Optimally, DNA typing could be performed prior to drug prescription in order to optimize therapy at the outset of psychotropic management. Those of skill in the art will be identify and associate these and other genes within each of the invention categories.

A preferred assessment table is provided below in Table 1.

| | Gene | | | | |
|---|---|---|---|---|---|
| Phenotype | (marker) | Outcome 1 | Outcome 2 | Outcome 3 | Outcome 4 |
| Codeine | CYP2D6 | Poor Met. | Intermediate | Extensive Met. | Ultrarapid Met. |
| | | | Met. | | |
| Tramadol | CYP2D6 | Poor Met. | Intermediate | Extensive Met. | Ultrarapid Met. |
| | | | Met. | | |
| Oxycodone | CYP2D6 | Poor Met. | Intermediate | Extensive Met. | Ultrarapid Met. |
| | | | Met. | | |
| Hydrocodone | CYP2D6 | Poor Met. | Intermediate | Extensive Met. | Ultrarapid Met. |
| | | | Met. | | |
| Methadone | CYP2B6 | Poor Met. | Intermediate | Extensive Met. | |
| | | | Met. | | |
| Fentanyl | OPRM1 | Decreased | Inconclusive | Typical efficacy | |
| | (rs1799971) | efficacy (G/G) | (G/A) | (A/A) | |

Diagnostic Methods

The invention further features diagnostic medicines, which are based, at least in part, on determination of the identity of the polymorphic region or expression level (or both in combination) of the genetic markers above.

For example, information obtained using the diagnostic assays described herein is useful for determining if a subject will respond to treatment for a given indication. Based on the prognostic information, a doctor can recommend a therapeutic protocol, useful for prescribing different treatment protocols for a given individual.

In addition, knowledge of the identity of a particular allele in an individual (the gene profile) allows customization of therapy for a particular disease to the individual's genetic profile, the goal of “pharmacogenomics”. For example, an individual's genetic profile can enable a doctor: 1) to more effectively prescribe a drug that will address the molecular basis of the disease or condition; 2) to better determine the appropriate dosage of a particular drug and 3) to identify novel targets for drug development. Expression patterns of individual patients can then be compared to the expression profile of the disease to determine the appropriate drug and dose to administer to the patient.

The ability to target populations expected to show the highest clinical benefit, based on the normal or disease genetic profile, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling.

Genotyping of an individual can be initiated before or after the individual begins to receive treatment.

“Palliating” a pain or one or more symptoms of a pain (such as rheumatoid arthritis pain or osteoarthritis pain) means lessening the extent of one or more undesirable I clinical manifestations of post-surgical pain in an individual or population of individuals treated with an analgesic in accordance with the invention.

Side effects of a particular treatment are those related to treatment based on a positive correlation between frequency or intensity of occurrence and drug treatment. Such information is usually collected in the course of studies on efficacy of a drug treatment and many methods are available to obtain such data. Resulting information is widely distributed among the medical profession and patients receiving treatment.

A treatment result is defined here from the point of view of the treating doctor, who judges the efficacy of a treatment as a group result. Within the group, individual patients can recover completely and some may even worsen, due to statistical variations in the course of the disease and the patient population. Some patients may discontinue treatment due to side effects, in which case no improvement in their condition due to analgesic treatment can occur. An improved treatment result is an overall improvement assessed over the whole group.

Improvement can be solely due to an overall reduction in frequency or intensity of side effects. It is also possible that doses can be increased or the dosing regime can be stepped up faster thanks to less troublesome side effects in the group and consequently an earlier onset of recovery or better remission of the disease.

A disorder, which is responsive to treatment with a particular drug or treatment, is defined to be a disorder, which is, according to recommendations in professional literature and drug formularies, known to respond with at least partial remission of the symptoms to a treatment with such drug or treatment. In most countries such recommendations are subject to governmental regulations, allowing and restricting the mention of medical indications in package inserts. Other sources are drug formularies of health management organizations. Before approval by governmental agencies certain recommendations can also be recognized by publications of confirmed treatment results in peer reviewed medical journals. Such collective body of information defines what is understood here to be a disorder that is responsive to treatment with an particular medication. Being responsive to particular treatment does not exclude that the disorder in an individual patient can resist treatment with such treatment, as long as a substantial portion of persons having the disorder respond with improvement to the treatment.

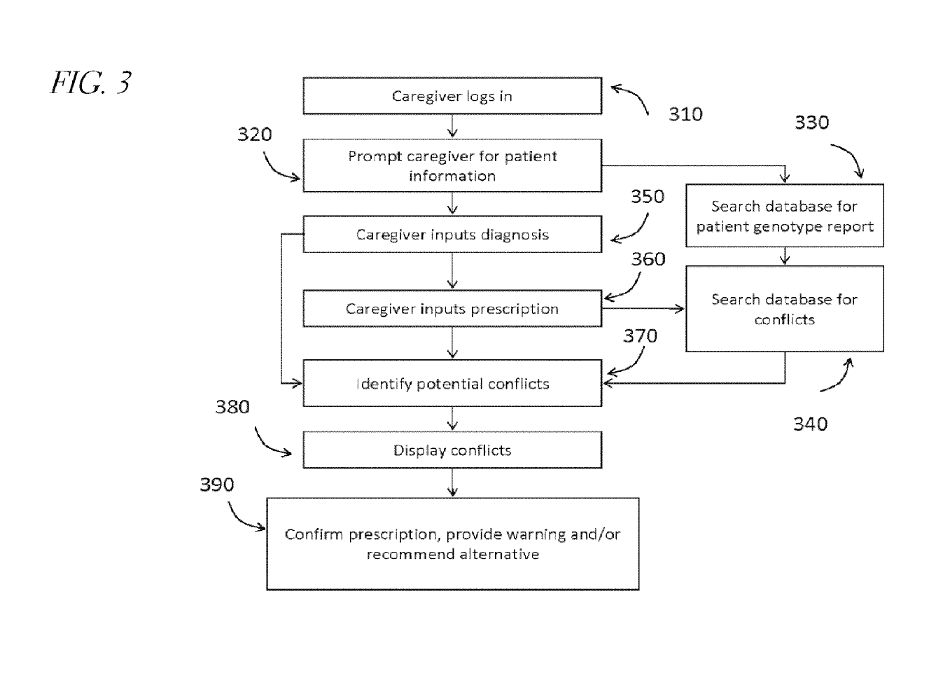

In a particular embodiment of the present invention, there are provided a method and system for healthcare providers (e.g., caregiver, physicians, doctors, nurses, pharmacists, insurance companies, therapist, medical specialists such as psychiatrists, etc.), or other to access information about the genetic profile of an individual to recommend or warn about particular treatments. FIG. 3 displays an interactive process of a healthcare provider, or individual with the invention system for recommending particular medications. A caregiver can access information 310 of their patient by accessing the system and interacting with the patient genetic records. As the system is targeted to providing personal information, the system will require the identity of the individual 320 to analyze or report upon. This information may be accessed 330 through information stored onsite or offsite in, for example, a patient data warehouse or with a laboratory or company providing such services. Either the system and/or the caregiver can provide additional information such as the diagnosis 350 (e.g., the genotyping may consist of analyzing an individual to detect genetic anomalies associated with the disorder or disease). Further, the caregiver can input any recommended prescriptions 360 that can be analyzed 340 against the individual's genetic profile to determine the efficacy and/or risk of such a treatment protocol.

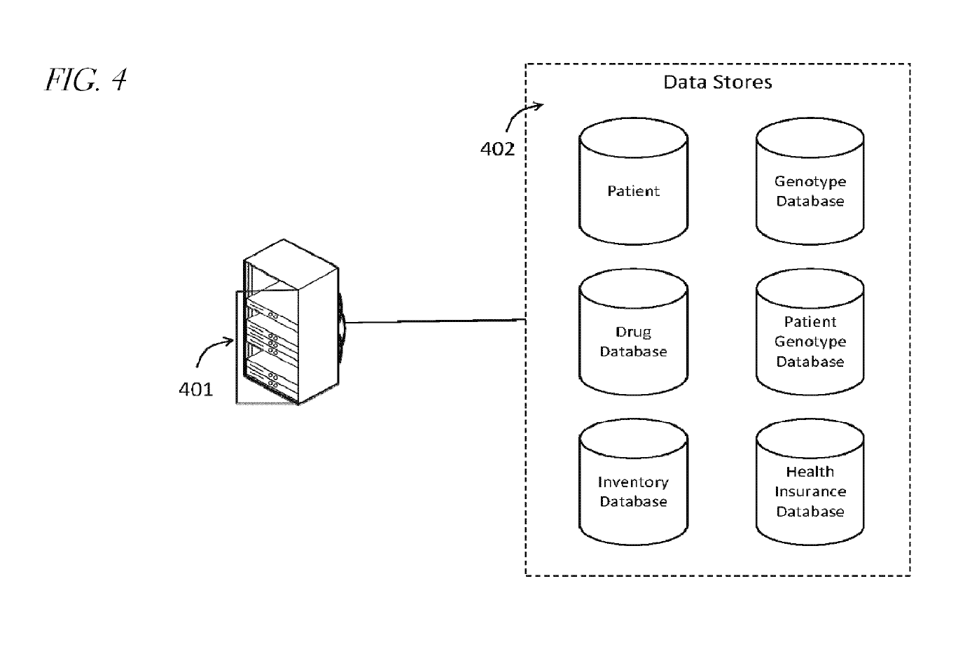

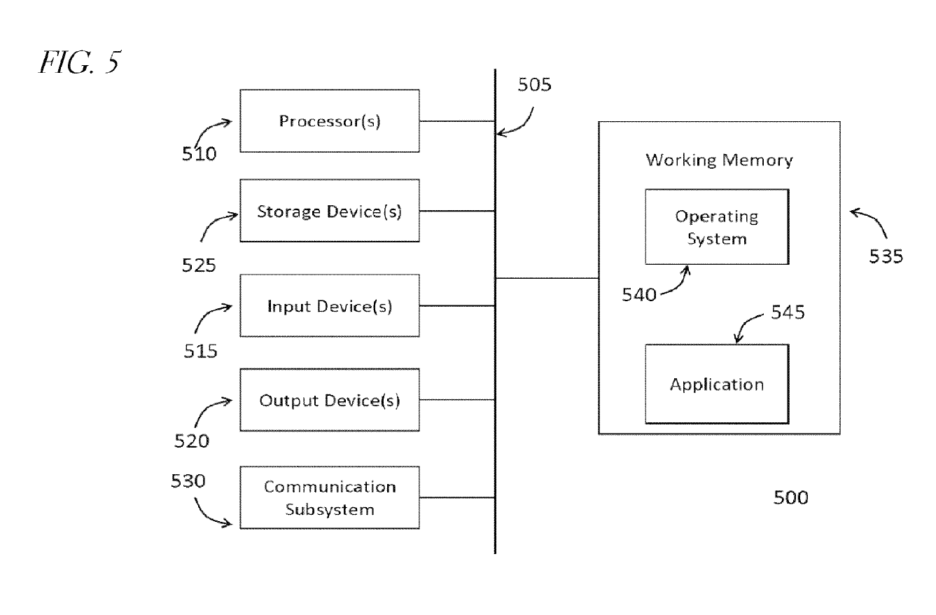

Any potential conflicts and problems can be flagged 370 and displayed 380 for the caregiver to review. Alternatively, the system can recommend or warn against particular medications and treatments, or classes of medications or treatments upon analysis of the individual's genetic profile as set forth in FIG. 7. Once any warnings or recommendations are made, the system can further confirm the determination of the caregiver, provide additional warnings or alternative medications or treatments 390. The system 401 can be tied, as shown in FIG. 4, into one or more additional databases 402 to further analyze inventory, price, insurance restrictions, treatment plans and the like.

Various embodiments of the invention provide for methods for identifying a genetic variation (e.g, allelic patterns, polymorphism patterns such as SNPs, or haplotype patterns etc.), comprising collecting biological samples from one or more subjects and exposing the samples to detection assays under conditions such that the presence or absence of at least one genetic variation is revealed. To begin, polynucleotide samples derived from (e.g., obtained from) an individual may be employed. Any biological sample that comprises a polynucleotide from the individual is suitable for use in the methods of the invention. The biological sample may be processed so as to isolate the polynucleotide. Alternatively, whole cells or other biological samples may be used without isolation of the polynucleotides contained therein.

Detection of a genetic variation in a polynucleotide sample derived from an individual can be accomplished by any means known in the art, including, but not limited to, amplification of a sequence with specific primers; determination of the nucleotide sequence of the polynucleotide sample; hybridization analysis; single strand conformational polymorphism analysis; denaturing gradient gel electrophoresis; mismatch cleavage detection; and the like. Detection of a genetic variation can also be accomplished by detecting an alteration in the level of a mRNA transcript of the gene; aberrant modification of the corresponding gene, e.g., an aberrant methylation pattern; the presence of a non-wild-type splicing pattern of the corresponding mRNA; an alteration in the level of the corresponding polypeptide; determining the electrophoretic mobility of the allele or fragments thereof (e.g., fragments generated by endonuclease digestion), and/or an alteration in corresponding polypeptide activity.

In some embodiments, a subject can be genotyped for an allele, more preferably a polymorphism by collecting and assaying a biological sample of the patient to determine the nucleotide sequence of the gene at that polymorphism, the amino acid sequence encoded by the gene at that polymorphism, or the concentration of the expressed product, e.g., by using one or more genotyping reagents, such as but not limited to nucleic acid reagents, including primers, etc., which may or may not be labeled, amplification enzymes, buffers, etc. In certain embodiments, the target polymorphism will be detected at the protein level, e.g., by assaying for a polymorphic protein. In yet other embodiments, the target polymorphism will be detected at the nucleic acid level, e.g., by assaying for the presence of nucleic acid polymorphism, e.g., a single nucleotide polymorphism (SNP) that cause expression of the polymorphic protein. Any convenient protocol for assaying a sample for the above one or more target polymorphisms may be employed in the subject methods.

In general, nucleic acid is extracted from the biological sample using conventional techniques. The nucleic acid to be extracted from the biological sample may be DNA, or RNA, typically total RNA. Typically RNA is extracted if the genetic variation to be studied is situated in the coding sequence of a gene. Where RNA is extracted from the biological sample, the methods further comprise a step of obtaining cDNA from the RNA. This may be carried out using conventional methods, such as reverse transcription using suitable primers. Subsequent procedures are then carried out on the extracted DNA or the cDNA obtained from extracted RNA. The term DNA, as used herein, may include both DNA and cDNA.

In general the genetic variations to be tested are known and characterized, e.g. in terms of sequence. Therefore nucleic acid regions comprising the genetic variations may be obtained using methods known in the art.

In one aspect, DNA regions which contain the genetic variations to be identified (target DNA regions) are subjected to an amplification reaction in order to obtain amplification products that contain the genetic variations to be identified. Any suitable technique or method may be used for amplification. In general, the technique allows the (simultaneous) amplification of all the DNA sequences containing the genetic variations to be identified. In other words, where multiple genetic variations are to be analyzed, it is preferable to simultaneously amplify all of the corresponding target DNA regions (comprising the variations). Carrying out the amplification in a single step (or as few steps as possible) simplifies the method.

Analyzing a polynucleotide sample can be conducted in a number of ways. Preferably, the allele can optionally be subjected to an amplification step prior to performance of the detection step. Preferred amplification methods are selected from the group consisting of: the polymerase chain reaction (PCR), the ligase chain reaction (LCR), strand displacement amplification (SDA), cloning, and variations of the above (e.g. RT-PCR and allele specific amplification). A test nucleic acid sample can be amplified with primers that amplify a region known to comprise the target polymorphism(s), for example, from within the metabolic gene loci, either flanking the marker of interest (as required for PCR amplification) or directly overlapping the marker (as in allele specific oligonucleotide (ASO) hybridization). In a particularly preferred embodiment, the sample is hybridized with a set of primers, which hybridize 5′ and 3′ in a sense or antisense sequence to the vascular disease associated allele, and is subjected to a PCR amplification. Genomic DNA or mRNA can be used directly or indirectly, for example, to convert into cDNA. Alternatively, the region of interest can be cloned into a suitable vector and grown in sufficient quantity for analysis.

The nucleic acid may be amplified by conventional techniques, such as a polymerase chain reaction (PCR), to provide sufficient amounts for analysis. The use of the polymerase chain reaction is described in a variety of publications, including, e.g., “PCR Protocols (Methods in Molecular Biology)” (2010) Daniel J. Park, eds, (Humana Press, 3^{rd} ed. (2011); and Saunders N A & Lee, M A. Eds “Real-Time PCR: Advanced Technologies and Applications (Caister Academic Press (2013). Other methods for amplification of nucleic acids is ligase chain reaction (“LCR”), disclosed in European Application No. 320 308, isothermal amplification method, such as described in Walker et al., (Proc. Nat'l Acad. Sci. USA 89:392-396, 1992) or Strand Displacement Amplification or Repair Chain Reaction (RCR), transcription-based amplification systems (TAS), including nucleic acid sequence based amplification (NASBA) and 3SR. Kwoh et al., Proc. Nat'l Acad. Sci. USA 86:1173 (1989); Gingeras et al., PCT Application WO 88/10315, cyclic and non-cyclic synthesis of single-stranded RNA (“ssRNA”), ssDNA, and double-stranded DNA (dsDNA) (Davey et al., European Application No. 329 822 and Miller et al., PCT Application WO 89/06700, respectively) and di-nucleotide amplification (Wu et. al., Genomics 4:560 1989). Miller et al., PCT Application WO 89/06700 Alternative amplification methods include: self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197, PCT Application No. PCT/US87/00880), or any other nucleic acid amplification method (e.g., GB Application No. 2 202 328, and in PCT Application No. PCT/US89/01025), followed by the detection of the amplified molecules using techniques known to those of skill in the art. These detection schemes are useful for the detection of nucleic acid molecules if such molecules are present in very low numbers.

Once the region of interest has been amplified, the genetic variant of interest can be detected in the PCR product by nucleotide sequencing, by SSCP analysis, or any other method known in the art. In one embodiment, any of a variety of sequencing reactions known in the art can be used to directly sequence at least a portion of the gene of interest and detect allelic variants, e.g., mutations, by comparing the sequence of the sample sequence with the corresponding wild-type (control) sequence. Exemplary sequencing reactions include those based on techniques developed by Maxam and Gilbert (1997) Proc. Natl. Acad Sci, USA 74:560 or Sanger et al. (1977) Proc. Nat. Acad. Sci, 74:5463. It is also contemplated that any of a variety of automated sequencing procedures can be utilized when performing the subject assays (Biotechniques (1995) 19:448), including sequencing by mass spectrometry (see, for example, U.S. Pat. No. 5,547,835 and International Patent Application Publication Number WO94/16101, entitled DNA Sequencing by Mass Spectrometry by H. Koster; U.S. Pat. No. 5,547,835 and international patent application Publication No. WO 94/21822 entitled “DNA Sequencing by Mass Spectrometry Via Exonuclease Degradation” by H. Koster; U.S. Pat. No. 5,605,798 and International Patent Application No. PCT/US96/03651 entitled DNA Diagnostics Based on Mass Spectrometry by H. Koster; Cohen et al. (1996) Adv. Chromat. 36:127-162; and Griffin et al. (1993) Appl Biochem Bio. 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

The high demand for low-cost sequencing has driven the development of high-throughput sequencing (or next-generation sequencing) technologies that parallelize the sequencing process, producing thousands or millions of sequences concurrently. High-throughput sequencing including ultra-high-throughput sequencing technologies are intended to lower the cost of DNA sequencing beyond what is possible with standard dye-terminator methods. These methods include pyrosequencing, reversible dye-terminator (Bentley, D. R.; Balasubramanian, S.; Swerdlow, H. P.; Smith, G. P.; Milton, J.; Brown, C. G.; Hall, K. P.; Evers, D. J. et al. (2008). “Accurate whole human genome sequencing using reversible terminator chemistry”. Nature 456 (7218): 53-59), SOLiD sequencing using sequencing by ligation Valouev A, Ichikawa J, Tonthat T et al. (July 2008). “A high-resolution, nucleosome position map of C. elegans reveals a lack of universal sequence-dictated positioning”. Genome Res. 18 (7): 1051-6), ion semiconductor sequencing (Rusk N (2011). “Torrents of sequence”. Nat Meth 8 (1): 44-44), Heliscope (single molecule sequcning (Helicos Biosciences, Thompson, J F; Steinmann, K E (2010 October). “Single molecule sequencing with a HeliScope genetic analysis system.”. Current protocols in molecular biology/edited by Frederick M. Ausubel . . . [et al.] Chapter 7: Unit 7.10), single molecule real-time (SMRT) sequencing (Pacific Biosciences; M. J. Levene, J. Korlach, S. W. Turner, M. Foquet, H. G. Craighead, W. W. Webb, Zero-Mode Waveguides for Single-Molecule Analysis at high concentrations. Science. 299 (2003) 682-686), nanopore DNA sequencing (M. J. Levene, J. Korlach, S. W. Turner, M. Foquet, H. G. Craighead, W. W. Webb, Zero-Mode Waveguides for Single-Molecule Analysis at high concentrations. Science. 299 (2003) 682-686), hybridization sequencing (Hanna G J, Johnson V A, Kuritzkes D R et al. (1 Jul. 2000). “Comparison of Sequencing by Hybridization and Cycle Sequencing for Genotyping of Human Immunodeficiency Virus Type 1 Reverse Transcriptase”. J. Clin. Microbiol. 38 (7): 2715-21), mass spectrometry sequencing (J. R. Edwards, H. Ruparel, and J. Ju (2005). “Mass-spectrometry DNA sequencing”. Mutation Research 573 (1-2): 3-12), Sanger microfluidic sequencing (Ying-Ja Chen, Eric E. Roller and Xiaohua Huang (2010). “DNA sequencing by denaturation: experimental proof of concept with an integrated fluidic device”. Lab on Chip 10 (10): 1153-1159), microscopy-based techniques such as transmission electron microscopy DNA sequencing (Ying-Ja Chen, Eric E. Roller and Xiaohua Huang (2010). “DNA sequencing by denaturation: experimental proof of concept with an integrated fluidic device”. Lab on Chip 10 (10): 1153-1159), RNA polymerase (RNAP) (Pareek, C S; Smoczynski, R; Tretyn, A (2011 November). “Sequencing technologies and genome sequencing.”. Journal of applied genetics 52 (4): 413-35), in vitro virus high-throughput sequencing (Fujimori, S; Hirai, N; Ohashi, H; Masuoka, K; Nishikimi, A; Fukui, Y; Washio, T; Oshikubo, T; Yamashita, T; Miyamoto-Sato, E (2012). “Next-generation sequencing coupled with a cell-free display technology for high-throughput production of reliable interactome data.”. Scientific reports 2: 691), and the like.

In some embodiments of the present invention, variant sequences are detected using a PCR-based assay. In some embodiments, the PCR assay comprises the use of oligonucleotide primers that hybridize only to the variant or wild type allele (e.g., to the region of polymorphism or mutation). Both sets of primers are used to amplify a sample of DNA. If only the mutant primers result in a PCR product, then the patient has the mutant allele. If only the wild-type primers result in a PCR product, then the patient has the wild type allele.

In preferred embodiments of the present invention, variant sequences are detected using a hybridization assay. In a hybridization assay, the presence of absence of a given SNP or mutation is determined based on the ability of the DNA from the sample to hybridize to a complementary DNA molecule (e.g., a oligonucleotide probe). Parameters such as hybridization conditions, polymorphic primer length, and position of the polymorphism within the polymorphic primer may be chosen such that hybridization will not occur unless a polymorphism present in the primer(s) is also present in the sample nucleic acid. Those of ordinary skill in the art are well aware of how to select and vary such parameters. See, e.g., Saiki et al. (1986) Nature 324:163; and Saiki et al (1989) Proc. Natl. Acad. Sci. USA 86:6230.

Yet other sequencing methods are disclosed, e.g., in U.S. Pat. No. 5,580,732 entitled “Method of DNA Sequencing Employing A Mixed DNA-Polymer Chain Probe” and U.S. Pat. No. 5,571,676 entitled “Method For Mismatch-Directed In Vitro DNA Sequencing.”

In some cases, the presence of the specific allele in DNA from a subject can be shown by restriction enzyme analysis. For example, the specific nucleotide polymorphism can result in a nucleotide sequence comprising a restriction site that is absent from the nucleotide sequence of another allelic variant.

In a further embodiment, protection from cleavage agents (such as a nuclease, hydroxylamine or osmium tetroxide and with piperidine) can be used to detect mismatched bases in RNA/RNA DNA/DNA, or RNA/DNA heteroduplexes (see, e.g., Myers et al. (1985) Science 230:1242). In general, the technique of “mismatch cleavage” starts by providing heteroduplexes formed by hybridizing a control nucleic acid, which is optionally labeled, e.g., RNA or DNA, comprising a nucleotide sequence of the allelic variant of the gene of interest with a sample nucleic acid, e.g., RNA or DNA, obtained from a tissue sample. The double-stranded duplexes are treated with an agent which cleaves single-stranded regions of the duplex such as duplexes formed based on basepair mismatches between the control and sample strands. For instance, RNA/DNA duplexes can be treated with RNase and DNA/DNA hybrids treated with 51 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine whether the control and sample nucleic acids have an identical nucleotide sequence or in which nucleotides they are different. See, for example, U.S. Pat. No. 6,455,249, Cotton et al. (1988) Proc. Natl. Acad. Sci. USA 85:4397; Saleeba et al. (1992) Methods Enzy. 217:286-295. In another embodiment, the control or sample nucleic acid is labeled for detection.

Over or under expression of a gene, in some cases, is correlated with a genomic polymorphism. The polymorphism can be present in an open reading frame (coded) region of the gene, in a “silent” region of the gene, in the promoter region, or in the 3′untranslated region of the transcript. Methods for determining polymorphisms are well known in the art and include, but are not limited to, the methods discussed below.

Detection of point mutations or additional base pair repeats (as required for the polymorphism) can be accomplished by molecular cloning of the specified allele and subsequent sequencing of that allele using techniques known in the art. Alternatively, the gene sequences can be amplified directly from a genomic DNA preparation from the sample using PCR, and the sequence composition is determined from the amplified product. As described more fully below, numerous methods are available for analyzing a subject's DNA for mutations at a given genetic locus such as the gene of interest.

A detection method is allele specific hybridization using probes overlapping the polymorphic site and having about 5, or alternatively 10, or alternatively 20, or alternatively 25, or alternatively 30 nucleotides around the polymorphic region. In another embodiment of the invention, several probes capable of hybridizing specifically to the allelic variant are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244.

Alternatively, various methods are known in the art that utilize oligonucleotide ligation as a means of detecting polymorphisms. See, e.g., Riley et al. (1990) Nucleic Acids Res. 18:2887-2890; and Delahunty et al. (1996) Am. J. Hum. Genet. 58:1239-1246.

In other embodiments, alterations in electrophoretic mobility are used to identify the particular allelic variant. For example, single strand conformation polymorphism (SSCP) may be used to detect differences in electrophoretic mobility between mutant and wild type nucleic acids (Orita et al. (1989) Proc Natl. Acad. Sci. USA 86:2766; Cotton (1993) Mutat. Res. 285:125-144 and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet. 7:5).

In performing SSCP analysis, the PCR product may be digested with a restriction endonuclease that recognizes a sequence within the PCR product generated by using as a template a reference sequence, but does not recognize a corresponding PCR product generated by using as a template a variant sequence by virtue of the fact that the variant sequence no longer contains a recognition site for the restriction endonuclease.

In yet another embodiment, the identity of the allelic variant is obtained by analyzing the movement of a nucleic acid comprising the polymorphic region in polyacrylamide gels containing a gradient of denaturant, which is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al. (1989) Proc. Natl. Acad. Sci. USA 86:6230 and Wallace et al. (1979) Nucl. Acids Res. 6:3543). Such allele specific oligonucleotide hybridization techniques may be used for the detection of the nucleotide changes in the polymorphic region of the gene of interest. For example, oligonucleotides having the nucleotide sequence of the specific allelic variant are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used in conjunction with the instant invention. Oligonucleotides used as primers for specific amplification may carry the allelic variant of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent, or reduce polymerase extension (Prossner (1993) Tibtech 11:238 and Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al. (1992) Mol. Cell. Probes 6:1).

In another embodiment, identification of the allelic variant is carried out using an oligonucleotide ligation assay (OLA), as described, e.g., in U.S. Pat. No. 4,998,617 and in Landegren, U. et al. Science 241:1077-1080 (1988). The OLA protocol uses two oligonucleotides that are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson et al. (1990) Proc. Natl. Acad. Sci. (U.S.A.) 87:8923-8927). In this method, PCR is used to achieve the exponential amplification of target DNA, which is then detected using OLA.

Several techniques based on this OLA method have been developed and can be used to detect the specific allelic variant of the polymorphic region of the gene of interest. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. (1996) Nucleic Acids Res. 24: 3728, OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

In one embodiment, the single base polymorphism can be detected by using a specialized exonuclease-resistant nucleotide, as disclosed, e.g., in Mundy (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of the polymorphic site. Cohen et al. (French Patent 2,650,840; PCT Appin. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet et al. (PCT Appin. No. 92/15712). This method uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appin. No. WO91/02087) the method of Goelet et al. supra, is preferably a heterogeneous phase assay, in which the primer or the target molecule is immobilized to a solid phase.

Recently, several primer-guided nucleotide incorporation procedures for assaying polymorphic sites in DNA have been described (Komher et al. (1989) Nucl. Acids. Res. 17:7779-7784; Sokolov (1990) Nucl. Acids Res. 18:3671; Syvanen et al. (1990) Genomics 8:684-692; Kuppuswamy et al. (1991) Proc. Natl. Acad. Sci. (U.S.A.) 88:1143-1147; Prezant et al. (1992) Hum. Mutat. 1:159-164; Ugozzoli et al. (1992) GATA 9:107-112; Nyren et al. (1993) Anal. Biochem. 208:171-175). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen et al. (1993) Amer. J. Hum. Genet. 52:46-59).

In one aspect the invention provided for a panel of genetic markers selected from, but not limited to the genetic polymorphisms above. The panel comprises probes or primers that can be used to amplify and/or for determining the molecular structure of the polymorphisms identified above. The probes or primers can be attached or supported by a solid phase support such as, but not limited to a gene chip or microarray. The probes or primers can be detectably labeled. This aspect of the invention is a means to identify the genotype of a patient sample for the genes of interest identified above. In one aspect, the methods of the invention provided for a means of using the panel to identify or screen patient samples for the presence of the genetic marker identified herein. In one aspect, the various types of panels provided by the invention include, but are not limited to, those described herein. In one aspect, the panel contains the above identified probes or primers as wells as other, probes or primers. In an alternative aspect, the panel includes one or more of the above noted probes or primers and others. In a further aspect, the panel consist only of the above-noted probes or primers.

In one embodiment of the invention, probes are labeled with two fluorescent dye molecules to form so-called “molecular beacons” (Tyagi and Kramer (1996) Nat. Biotechnol. 14:303-8). Such molecular beacons signal binding to a complementary nucleic acid sequence through relief of intramolecular fluorescence quenching between dyes bound to opposing ends on an oligonucleotide probe. The use of molecular beacons for genotyping has been described (Kostrikis (1998) Science 279:1228-9) as has the use of multiple beacons simultaneously (Marras (1999) Genet. Anal. 14:151-6). A quenching molecule is useful with a particular fluorophore if it has sufficient spectral overlap to substantially inhibit fluorescence of the fluorophore when the two are held proximal to one another, such as in a molecular beacon, or when attached to the ends of an oligonucleotide probe from about 1 to about 25 nucleotides.

Labeled probes also can be used in conjunction with amplification of a polymorphism. (Holland et al. (1991) Proc. Natl. Acad. Sci. 88:7276-7280). U.S. Pat. No. 5,210,015 by Gelfand et al. describe fluorescence-based approaches to provide real time measurements of amplification products during PCR. Such approaches have either employed intercalating dyes (such as ethidium bromide) to indicate the amount of double-stranded DNA present, or they have employed probes containing fluorescence-quencher pairs (also referred to as the “Taq-Man” approach) where the probe is cleaved during amplification to release a fluorescent molecule whose concentration is proportional to the amount of double-stranded DNA present. During amplification, the probe is digested by the nuclease activity of a polymerase when hybridized to the target sequence to cause the fluorescent molecule to be separated from the quencher molecule, thereby causing fluorescence from the reporter molecule to appear. The Taq-Man approach uses a probe containing a reporter molecule—quencher molecule pair that specifically anneals to a region of a target polynucleotide containing the polymorphism.

Probes can be affixed to surfaces for use as “gene chips” or “microarray.” Such gene chips or microarrays can be used to detect genetic variations by a number of techniques known to one of skill in the art. In one technique, oligonucleotides are arrayed on a gene chip for determining the DNA sequence of a by the sequencing by hybridization approach, such as that outlined in U.S. Pat. Nos. 6,025,136 and 6,018,041. The probes of the invention also can be used for fluorescent detection of a genetic sequence. Such techniques have been described, for example, in U.S. Pat. Nos. 5,968,740 and 5,858,659. A probe also can be affixed to an electrode surface for the electrochemical detection of nucleic acid sequences such as described by Kayem et al. U.S. Pat. No. 5,952,172 and by Kelley et al. (1999) Nucleic Acids Res. 27:4830-4837.

Various “gene chips” or “microarray” and similar technologies are known in the art. Examples of such include, but are not limited to LabCard (ACLARA Bio Sciences Inc.); GeneChip (Affymetrix, Inc); LabChip (Caliper Technologies Corp); a low-density array with electrochemical sensing (Clinical Micro Sensors); LabCD System (Gamera Bioscience Corp.); Omni Grid (Gene Machines); Q Array (Genetix Ltd.); a high-throughput, automated mass spectrometry systems with liquid-phase expression technology (Gene Trace Systems, Inc.); a thermal jet spotting system (Hewlett Packard Company); Hyseq HyChip (Hyseq, Inc.); BeadArray (Illumina, Inc., San Diego WO 99/67641 and WO 00/39587); GEM (Incyte Microarray Systems); a high-throughput microarraying system that can dispense from 12 to 64 spots onto multiple glass slides (Intelligent Bio-Instruments); Molecular Biology Workstation and NanoChip (Nanogen, Inc.); a microfluidic glass chip (Orchid biosciences, Inc.); surface tension array (ProtoGene, Palo Alto, Calif. U.S. Pat. Nos. 6,001,311; 5,985,551; and 5,474,796), BioChip Arrayer with four PiezoTip piezoelectric drop-on-demand tips (Packard Instruments, Inc.); FlexJet (Rosetta Inpharmatic, Inc.); MALDI-TOF mass spectrometer (Sequnome); ChipMaker 2 and ChipMaker 3 (TeleChem International, Inc.); and GenoSensor (Vysis, Inc.) as identified and described in Heller (2002) Annu Rev. Biomed. Eng. 4:129-153. Examples of “Gene chips” or a “microarray” are also described in US Patent Publ. Nos.: 2007-0111322, 2007-0099198, 2007-0084997, 2007-0059769 and 2007-0059765 and U.S. Pat. Nos. 7,138,506, 7,070,740, and 6,989,267.

In one aspect, “gene chips” or “microarrays” containing probes or primers for genes of the invention alone or in combination are prepared. A suitable sample is obtained from the patient extraction of genomic DNA, RNA, or any combination thereof and amplified if necessary. The DNA or RNA sample is contacted to the gene chip or microarray panel under conditions suitable for hybridization of the gene(s) of interest to the probe(s) or primer(s) contained on the gene chip or microarray. The probes or primers may be detectably labeled thereby identifying the polymorphism in the gene(s) of interest. Alternatively, a chemical or biological reaction may be used to identify the probes or primers which hybridized with the DNA or RNA of the gene(s) of interest. The genotypes of the patient is then determined with the aid of the aforementioned apparatus and methods.

An allele may also be detected indirectly, e.g. by analyzing the protein product encoded by the DNA. For example, where the marker in question results in the translation of a mutant protein, the protein can be detected by any of a variety of protein detection methods. Such methods include immunodetection and biochemical tests, such as size fractionation, where the protein has a change in apparent molecular weight either through truncation, elongation, altered folding or altered post-translational modifications. Methods for measuring gene expression are also well known in the art and include, but are not limited to, immunological assays, nuclease protection assays, northern blots, in situ hybridization, reverse transcriptase Polymerase Chain Reaction (RT-PCR), Real-Time Polymerase Chain Reaction, expressed sequence tag (EST) sequencing, cDNA microarray hybridization or gene chip analysis, statistical analysis of microarrays (SAM), subtractive cloning, Serial Analysis of Gene Expression (SAGE), Massively Parallel Signature Sequencing (MPSS), and Sequencing-By-Synthesis (SBS). See for example, Carulli et al., (1998) J. Cell. Biochem. 72 (S30-31): 286-296; Galante et al., (2007) Bioinformatics, Advance Access (Feb. 3, 2007).

SAGE, MPSS, and SBS are non-array based assays that determine the expression level of genes by measuring the frequency of sequence tags derived from polyadenylated transcripts. SAGE allows for the analysis of overall gene expression patterns with digital analysis. SAGE does not require a preexisting clone and can used to identify and quantitate new genes as well as known genes. Velculescu et al., (1995) Science 270(5235):484-487; Velculescu (1997) Cell 88(2):243-251.

MPSS technology allows for analyses of the expression level of virtually all genes in a sample by counting the number of individual mRNA molecules produced from each gene. As with SAGE, MPSS does not require that genes be identified and characterized prior to conducting an experiment. MPSS has a sensitivity that allows for detection of a few molecules of mRNA per cell. Brenner et al. (2000) Nat. Biotechnol. 18:630-634; Reinartz et al., (2002) Brief Funct. Genomic Proteomic 1: 95-104.

SBS allows analysis of gene expression by determining the differential expression of gene products present in sample by detection of nucleotide incorporation during a primer-directed polymerase extension reaction.

SAGE, MPSS, and SBS allow for generation of datasets in a digital format that simplifies management and analysis of the data. The data generated from these analyses can be analyzed using publicly available databases such as Sage Genie (Boon et al., (2002) PNAS 99:11287-92), SAGEmap (Lash et al., (2000) Genome Res 10:1051-1060), and Automatic Correspondence of Tags and Genes (ACTG) (Galante (2007), supra). The data can also be analyzed using databases constructed using in house computers (Blackshaw et al. (2004) PLoS Biol, 2:E247; Silva et al. (2004) Nucleic Acids Res 32:6104-6110)).

Moreover, it will be understood that any of the above methods for detecting alterations in a gene or gene product or polymorphic variants can be used to monitor the course of treatment or therapy.

The methods described herein may be performed, for example, by utilizing pre-packaged diagnostic kits, such as those described below, comprising at least one probe or primer nucleic acid described herein, which may be conveniently used, e.g., to determine whether a subject has or may have a greater or lower response to analgesic treatments.

Diagnostic procedures can also be performed in situ directly upon samples from, such that no nucleic acid purification is necessary. Nucleic acid reagents can be used as probes and/or primers for such in situ procedures (see, for example, Nuovo (1992) “PCR 1N SITU HYBRIDIZATION: PROTOCOLS AND APPLICATIONS”, Raven Press, NY).

In addition to methods that focus primarily on the detection of one nucleic acid sequence, profiles can also be assessed in such detection schemes. Fingerprint profiles can be generated, for example, by utilizing a differential display procedure, Northern analysis and/or RT-PCR.

### Nucleic Acids

In one aspect, the nucleic acid sequences of the gene's allelic variants, or portions thereof, can be the basis for probes or primers, e.g., in methods and compositions for determining and identifying the allele present at the gene of interest's locus, more particularly to identity the allelic variant of a polymorphic region(s). Thus, they can be used in the methods of the invention to determine which therapy is most likely to affect or not affect an individual's disease or disorder, such as to diagnose and prognoses disease progression as well as select the most effective treatment among treatment options. Probes can be used to directly determine the genotype of the sample or can be used simultaneously with or subsequent to amplification.

The methods of the invention can use nucleic acids isolated from vertebrates. In one aspect, the vertebrate nucleic acids are mammalian nucleic acids. In a further aspect, the nucleic acids used in the methods of the invention are human nucleic acids.

Primers and probes for use in the methods of the invention are nucleic acids that hybridize to a nucleic acid sequence which is adjacent to the region of interest or which covers the region of interest and is extended. A primer or probe can be used alone in a detection method, or a can be used together with at least one other primer or probe in a detection method. Primers can also be used to amplify at least a portion of a nucleic acid. Probes for use in the methods of the invention are nucleic acids which hybridize to the region of interest and which are generally are not further extended. Probes may be further labeled, for example by nick translation, Klenow fill-in reaction, PCR or other methods known in the art, including those described herein). For example, a probe is a nucleic acid which hybridizes to the polymorphic region of the gene of interest, and which by hybridization or absence of hybridization to the DNA of a subject will be indicative of the identity of the allelic variant of the polymorphic region of the gene of interest. Probes and primers of the present invention, their preparation and/or labeling are described in Green and Sambrook (2012). Primers and Probes useful in the methods described herein are found in Table 1.

In one embodiment, primers and probes comprise a nucleotide sequence which comprises a region having a nucleotide sequence which hybridizes under stringent conditions to about 5 through about 100 consecutive nucleotides, more particularly about: 6, 8, 10, 12, 15, 20, 25, 30, 35, 40, 45, 50, 60, or 75 consecutive nucleotides of the gene of interest. Length of the primer or probe used will depend, in part, on the nature of the assay used and the hybridization conditions employed.

Primers can be complementary to nucleotide sequences located close to each other or further apart, depending on the use of the amplified DNA. For example, primers can be chosen such that they amplify DNA fragments of at least about 10 nucleotides or as much as several kilobases. Preferably, the primers of the invention will hybridize selectively to nucleotide sequences located about 150 to about 350 nucleotides apart.

For amplifying at least a portion of a nucleic acid, a forward primer (i.e., 5′ primer) and a reverse primer (i.e., 3′ primer) will preferably be used. Forward and reverse primers hybridize to complementary strands of a double stranded nucleic acid, such that upon extension from each primer, a double stranded nucleic acid is amplified.

Yet other preferred primers of the invention are nucleic acids that are capable of selectively hybridizing to an allelic variant of a polymorphic region of the gene of interest. Thus, such primers can be specific for the gene of interest sequence, so long as they have a nucleotide sequence that is capable of hybridizing to the gene of interest.

The probe or primer may further comprises a label attached thereto, which, e.g., is capable of being detected, e.g. the label group is selected from amongst radioisotopes, fluorescent compounds, enzymes, and enzyme co-factors.

Additionally, the isolated nucleic acids used as probes or primers may be modified to become more stable. Exemplary nucleic acid molecules that are modified include phosphoramidate, phosphothioate and methylphosphonate analogs of DNA (see also U.S. Pat. Nos. 5,176,996; 5,264,564 and 5,256,775).

The nucleic acids used in the methods of the invention can also be modified at the base moiety, sugar moiety, or phosphate backbone, for example, to improve stability of the molecule. The nucleic acids, e.g., probes or primers, may include other appended groups such as peptides (e.g., for targeting host cell receptors in vivo), or agents facilitating transport across the cell membrane. See, e.g., Letsinger et al., (1989) Proc. Natl. Acad. Sci. U.S.A. 86:6553-6556; Lemaitre et al., (1987) Proc. Natl. Acad. Sci. 84:648-652; and PCT Publication No. WO 88/09810, published Dec. 15, 1988), hybridization-triggered cleavage agents, (see, e.g., Krol et al., (1988) BioTechniques 6:958-976) or intercalating agents (see, e.g., Zon (1988) Pharm. Res. 5:539-549. To this end, the nucleic acid used in the methods of the invention may be conjugated to another molecule, e.g., a peptide, hybridization triggered cross-linking agent, transport agent, hybridization-triggered cleavage agent, etc.

The isolated nucleic acids used in the methods of the invention can also comprise at least one modified sugar moiety selected from the group including but not limited to arabinose, 2-fluoroarabinose, xylulose, and hexose or, alternatively, comprise at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

The nucleic acids, or fragments thereof, to be used in the methods of the invention can be prepared according to methods known in the art and described, e.g., in Sambrook and Russel (2001) supra. For example, discrete fragments of the DNA can be prepared and cloned using restriction enzymes. Alternatively, discrete fragments can be prepared using the Polymerase Chain Reaction (PCR) using primers having an appropriate sequence under the manufacturer's conditions, (described above).

Oligonucleotides can be synthesized by standard methods known in the art, e.g. by use of an automated DNA synthesizer (such as are commercially available from Biosearch, Applied Biosystems, etc.). As examples, phosphorothioate oligonucleotides can be synthesized by the method of Stein et al. (1988) Nucl. Acids Res. 16:3209, methylphosphonate oligonucleotides can be prepared by use of controlled pore glass polymer supports. Sarin et al. (1988) Proc. Natl. Acad. Sci. U.S.A. 85:7448-7451.

### Kits

As set forth herein, the invention provides diagnostic methods for determining the type of allelic variant of a polymorphic region present in the gene of interest or the expression level of a gene of interest. In some embodiments, the methods use probes or primers comprising nucleotide sequences which are complementary to the polymorphic region of the gene of interest. Accordingly, the invention provides kits for performing these methods as well as instructions for carrying out the methods of this invention such as collecting tissue and/or performing the screen, and/or analyzing the results, and/or administration of an effective amount of the therapies described above.

In an embodiment, the invention provides a kit for determining whether a subject responds to analgesic treatment or alternatively one of various treatment options. The kits contain one of more of the compositions described above and instructions for use. As an example only, the invention also provides kits for determining response to analgesic treatment containing a first and a second oligonucleotide specific for the polymorphic region of the gene. Oligonucleotides “specific for” a genetic locus bind either to the polymorphic region of the locus or bind adjacent to the polymorphic region of the locus. For oligonucleotides that are to be used as primers for amplification, primers are adjacent if they are sufficiently close to be used to produce a polynucleotide comprising the polymorphic region. In one embodiment, oligonucleotides are adjacent if they bind within about 1-2 kb, and preferably less than 1 kb from the polymorphism. Specific oligonucleotides are capable of hybridizing to a sequence, and under suitable conditions will not bind to a sequence efficiently differing by a single nucleotide.

The kit can comprise at least one probe or primer which is capable of specifically hybridizing to the polymorphic region of the gene of interest and instructions for use. The kits preferably comprise at least one of the above described nucleic acids. Preferred kits for amplifying at least a portion of the gene of interest comprise two primers and two probes, at least one of probe is capable of binding to the allelic variant sequence. Such kits are suitable for detection of genotype by, for example, fluorescence detection, by electrochemical detection, or by other detection.

Oligonucleotides, whether used as probes or primers, contained in a kit can be detectably labeled. Labels can be detected either directly, for example for fluorescent labels, or indirectly. Indirect detection can include any detection method known to one of skill in the art, including biotin-avidin interactions, antibody binding and the like. Fluorescently labeled oligonucleotides also can contain a quenching molecule. Oligonucleotides can be bound to a surface. In one embodiment, the preferred surface is silica or glass. In another embodiment, the surface is a metal electrode.

Yet other kits of the invention comprise at least one reagent necessary to perform the assay. For example, the kit can comprise an enzyme. Alternatively the kit can comprise a buffer or any other necessary reagent.

Conditions for incubating a nucleic acid probe with a test sample depend on the format employed in the assay, the detection methods used, and the type and nature of the nucleic acid probe used in the assay. One skilled in the art will recognize that any one of the commonly available hybridization, amplification or immunological assay formats can readily be adapted to employ the nucleic acid probes for use in the present invention. Examples of such assays can be found in Chard (1986) AN INTRODUCTION TO RADIOIMMUNOASSAY AND RELATED TECHNIQUES Elsevier Science Publishers, Amsterdam, The Netherlands; Bullock et al. TECHNIQUES IN IMMUNOCYTOCHEMISTRY Academic Press, Orlando, Fla. Vol. 1 (1982), Vol. 2 (1983), Vol. 3 (1985); Tijssen, PRACTICE AND THEORY OF IMMUNOASSAYS: LABORATORY TECHNIQUES IN BIOCHEMISTRY AND MOLECULAR BIOLOGY, Elsevier Science Publishers, Amsterdam, The Netherlands (1985).

The test samples used in the diagnostic kits include cells, protein or membrane extracts of cells, or biological fluids such as sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are known in the art and can be readily adapted in order to obtain a sample which is compatible with the system utilized.

The kits can include all or some of the positive controls, negative controls, reagents, primers, sequencing markers, probes and antibodies described herein for determining the subject's genotype in the polymorphic region or the expression levels of the gene of interest.

As amenable, these suggested kit components may be packaged in a manner customary for use by those of skill in the art. For example, these suggested kit components may be provided in solution or as a liquid dispersion or the like.

### Other Uses for the Nucleic Acids of the Invention

The identification of the allele of the gene of interest can also be useful for identifying an individual among other individuals from the same species. For example, DNA sequences can be used as a fingerprint for detection of different individuals within the same species. Thompson and Thompson, Eds., (1991) GENETICS IN MEDICINE, W B Saunders Co., Philadelphia, Pa. This is useful, e.g., in forensic studies.

The invention now being generally described, it will be more readily understood by reference to the following examples which are included merely for purposes of illustration of certain aspects and embodiments of the present invention, and are not intended to limit the invention.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purpose of exemplification only. Functionally-equivalent products, compositions and methods are clearly within the scope of the invention, as described herein.

The present invention is performed without undue experimentation using, unless otherwise indicated, conventional techniques of molecular biology, microbiology, virology, recombinant DNA technology, peptide synthesis in solution, solid phase peptide synthesis, histology and immunology. Such procedures are described, for example, in the following texts that are incorporated by reference:
(i) Green M R, Sambrook J, Molecular Cloning: A Laboratory Manual, Cold Spring Harbor Laboratories Press, New York, Fourth Edition (2012), whole of Vols I, II, and III;
(ii) DNA Cloning: A Practical Approach, Vols. I-IV (D. M. Glover, ed., 1995), Oxford University Press, whole of text;
(iii) Oligonucleotide Synthesis: Methods and Application (P Herdewijn, ed., 2010) Humana Press, Oxford, whole of text;
(iv) Nucleic Acid Hybridization: A Practical Approach (B. D. Hames & S. J. Higgins, eds., 1985) IRL Press, Oxford, whole of text;
(v) van Pelt-Verkuil, E, van Belkum, A, Hays, J P. Principles and Technical Aspects of PCR Amplification (2010) Springer, whole of text;
(vi) Perbal, B., A Practical Guide to Molecular Cloning, 3rd Ed. (2008);
(vii) Gene Synthesis: Methods and Protocols (J Peccoud, ed. 2012) Humana Press, whole of text;
(viii) PCR Primer Design (Methods in Molecular Biology). (A Yuryev. ed., 2010), Humana Press, Oxford, whole of text.

### Computer Embodiment

FIG. 5 provides a schematic illustration of one embodiment of a computer system 1500 that can perform the methods of the invention, as described herein. It should be noted that FIG. 5 is meant only to provide a generalized illustration of various components, any or all of which may be utilized as appropriate. FIG. 5, therefore, broadly illustrates how individual system elements may be implemented in a relatively separated or relatively more integrated manner.

The computer system 500 is shown comprising hardware elements that can be electrically coupled via a bus 505 (or may otherwise be in communication, as appropriate). The hardware elements can include one or more processors 510, including without limitation, one or more general purpose processors and/or one or more special purpose processors (such as digital signal processing chips, graphics acceleration chips, and/or the like); one or more input devices 515, which can include without limitation a mouse, a keyboard and/or the like; and one or more output devices 520, which can include without limitation a display device, a printer and/or the like.

The computer system 500 may further include (and/or be in communication with) one or more storage devices 525, which can comprise, without limitation, local and/or network accessible storage and/or can include, without limitation, a disk drive, a drive array, an optical storage device, a solid state storage device such as a random access memory (“RAM”) and/or a read-only memory (“ROM”), which can be programmable, flash updateable and/or the like. The computer system 500 might also include a communications subsystem 530, which can include without limitation a modem, a network card (wireless or wired), an infrared communication device, a wireless communication device and/or chipset (such as a Bluetooth™ device, an 802.11 device, a WiFi device, a WiMax device, cellular communication facilities, etc.), and/or the like. The communications subsystem 530 may permit data to be exchanged with a network (such as the network described below, to name one example), and/or any other devices described herein. In many embodiments, the computer system 500 will further comprise a working memory 535, which can include a RAM or ROM device, as described above.

The computer system 500 also can comprise software elements, shown as being currently located within the working memory 535, including an operating system 540 and/or other code, such as one or more application programs 545, which may comprise computer programs of the invention, and/or may be designed to implement methods of the invention and/or configure systems of the invention, as described herein. Merely by way of example, one or more procedures described with respect to the method(s) discussed above might be implemented as code and/or instructions executable by a computer (and/or a processor within a computer). A set of these instructions and/or codes might be stored on a computer-readable storage medium, such as the storage device(s) 525 described above. In some cases, the storage medium might be incorporated within a computer system, such as the system 500. In other embodiments, the storage medium might be separate from a computer system (i.e., a removable medium, such as a compact disc, etc.), and is provided in an installation package, such that the storage medium can be used to program a general-purpose computer with the instructions/code stored therein. These instructions might take the form of executable code, which is executable by the computer system 500 and/or might take the form of source and/or installable code, which, upon compilation and/or installation on the computer system 500 (e.g., using any of a variety of generally available compilers, installation programs, compression/decompression utilities, etc.), then takes the form of executable code.

It will be apparent to those skilled in the art that substantial variations may be made in accordance with specific requirements. For example, customized hardware might also be used, and/or particular elements might be implemented in hardware, software (including portable software, such as applets, etc.), or both. Further, connection to other computing devices such as network input/output devices may be employed.

In one aspect, the invention employs a computer system (such as the computer system 500) to perform methods of the invention. According to a set of embodiments, some or all of the procedures of such methods are performed by the computer system 500 in response to processor 510 executing one or more sequences of one or more instructions (which might be incorporated into the operating system 540 and/or other code, such as an application program 545) contained in the working memory 535. Such instructions may be read into the working memory 535 from another machine-readable medium, such as one or more of the storage device(s) 525. Merely by way of example, execution of the sequences of instructions contained in the working memory 535 might cause the processor(s) 510 to perform one or more procedures of the methods described herein.

The terms “machine-readable medium” and “computer readable medium,” as used herein, refer to any medium that participates in providing data that causes a machine to operate in a specific fashion. In an embodiment implemented using the computer system 500, various machine-readable media might be involved in providing instructions/code to processor(s) 510 for execution and/or might be used to store and/or carry such instructions/code (e.g., as signals). In many implementations, a computer-readable medium is a physical and/or tangible storage medium. Such a medium may take many forms, including but not limited to, non-volatile media, volatile media, and transmission media. Non-volatile media includes, for example, optical or magnetic disks, such as the storage device(s) 525. Volatile media includes, without limitation, dynamic memory, such as the working memory 535. Transmission media includes coaxial cables, copper wire and fiber optics, including the wires that comprise the bus 505, as well as the various components of the communications subsystem 530 (and/or the media by which the communications subsystem 530 provides communication with other devices). Hence, transmission media can also take the form of waves (including without limitation radio, acoustic and/or light waves, such as those generated during radio wave and infrared data communications).

Common forms of physical and/or tangible computer-readable media include, for example, a floppy disk, a flexible disk, a hard disk, magnetic tape, or any other magnetic medium, a CD-ROM, any other optical medium, punchcards, papertape, any other physical medium with patterns of holes, a RAM, a PROM, an EPROM, a FLASH-EPROM, any other memory chip or cartridge, a carrier wave as described hereinafter, or any other medium from which a computer can read instructions and/or code.

Various forms of machine-readable media may be involved in carrying one or more sequences of one or more instructions to the processor(s) 510 for execution. Merely by way of example, the instructions may initially be carried on a magnetic disk and/or optical disc of a remote computer. A remote computer might load the instructions into its dynamic memory and send the instructions as signals over a transmission medium to be received and/or executed by the computer system 500. These signals, which might be in the form of electromagnetic signals, acoustic signals, optical signals and/or the like, are all examples of carrier waves on which instructions can be encoded, in accordance with various embodiments of the invention.

The communications subsystem 530 (and/or components thereof) generally will receive the signals, and the bus 505 then might carry the signals (and/or the data, instructions, etc., carried by the signals) to the working memory 535, from which the processor(s) 510 retrieves and executes the instructions. The instructions received by the working memory 535 may optionally be stored on a storage device 525 either before or after execution by the processor(s) 510.

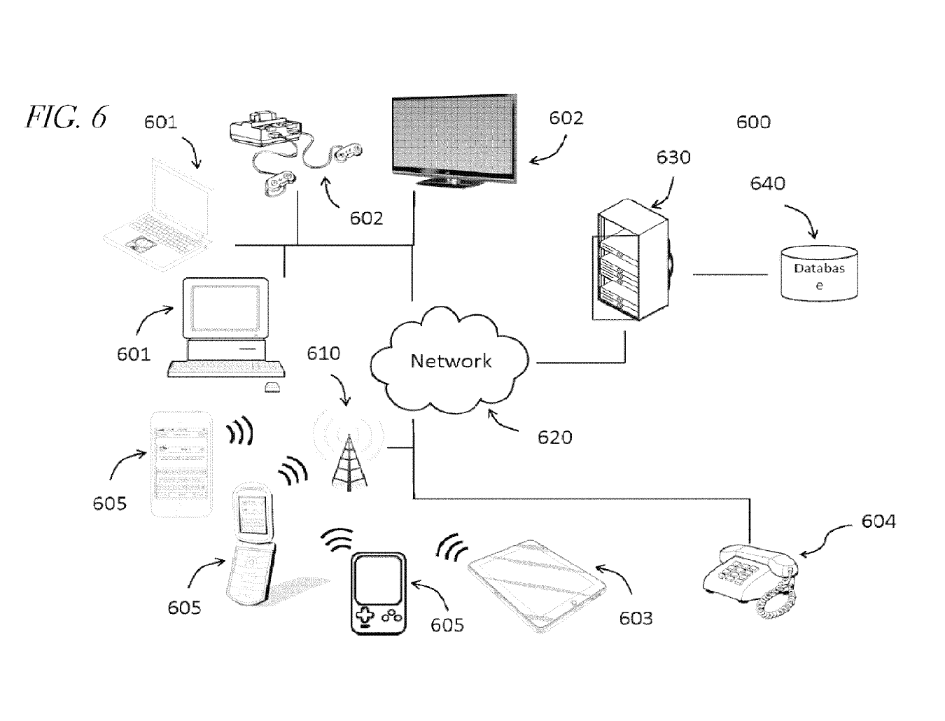

Merely by way of example, FIG. 6 illustrates a schematic diagram of devices to access and implement the invention system 600. The system 600 can include one or more user computers 601. The user computers 601 can be general-purpose personal computers (including, merely by way of example, personal computers and/or laptop computers running any appropriate flavor of Microsoft Corp.'s Windows™ and/or Apple Corp.'s Macintosh™ operating systems) and/or workstation computers running any of a variety of commercially available UNIX™ or UNIX-like operating systems. These user computers 601 can also have any of a variety of applications, including one or more applications configured to perform methods of the invention, as well as one or more office applications, database client and/or server applications, and web browser applications. Alternatively, the user computers 601 can be any other electronic device, such as a thin-client computer, media computing platforms 602 (e.g., gaming platforms, or cable and satellite set top boxes with navigation and recording capabilities), handheld computing devices (e.g., PDAs, tablets or handheld gaming platforms) 603, conventional land lines 604 (wired and wireless), mobile (e.g., cell or smart) phones 605 or tablets, or any other type of portable communication or computing platform (e.g., vehicle navigation systems), capable of communicating via a network (e.g., the network 620 described below) and/or displaying and navigating web pages or other types of electronic documents. Although the exemplary system 600 is shown with a user computer 601, any number of user computers can be supported.

Certain embodiments of the invention operate in a networked environment, which can include a network 620. The network 620 can be any type of network familiar to those skilled in the art that can support data communications using any of a variety of commercially available protocols, including without limitation TCP/IP, SNA, IPX, AppleTalk, and the like. Merely by way of example, the network 620 can be a local area network (“LAN”), including without limitation an Ethernet network, a Token-Ring network and/or the like; a wide-area network (WAN); a virtual network, including without limitation a virtual private network (“VPN”); the Internet; an intranet; an extranet; a public switched telephone network (“PSTN”); an infrared network; a wireless network 610, including without limitation a network operating under any of the IEEE 802.11 suite of protocols, the Bluetooth™ protocol known in the art, and/or any other wireless protocol 610; and/or any combination of these and/or other networks.

Embodiments of the invention can include one or more server computers 630. Each of the server computers 630 may be configured with an operating system, including without limitation any of those discussed above, as well as any commercially (or freely) available server operating systems. Each of the servers 630 may also be running one or more applications, which can be configured to provide services to one or more clients and/or other servers.

Merely by way of example, one of the servers 630 may be a web server, which can be used, merely by way of example, to process requests for web pages or other electronic documents from user computers 601. The web server can also run a variety of server applications, including HTTP servers, FTP servers, CGI servers, database servers, Java™ servers, and the like. In some embodiments of the invention, the web server may be configured to serve web pages that can be operated within a web browser on one or more of the user computers 601 to perform methods of the invention.

The server computers 630, in some embodiments, might include one or more application servers, which can include one or more applications accessible by a client running on one or more of the client computers and/or other servers. Merely by way of example, the server(s) 630 can be one or more general purpose computers capable of executing programs or scripts in response to the user computers and/or other servers, including without limitation web applications (which might, in some cases, be configured to perform methods of the invention). Merely by way of example, a web application can be implemented as one or more scripts or programs written in any suitable programming language, such as Java™, C, C#™ or C++, and/or any scripting language, such as Perl, Python, or TCL, as well as combinations of any programming/scripting languages. The application server(s) can also include database servers, including without limitation those commercially available from Oracle™, Microsoft™ Sybase™, IBM™ and the like, which can process requests from clients (including, depending on the configuration, database clients, API clients, web browsers, etc.) running on a user computer and/or another server. In some embodiments, an application server can create web pages dynamically for displaying the information in accordance with embodiments of the invention. Data provided by an application server may be formatted as web pages (comprising HTML, Javascript, etc., for example) and/or may be forwarded to a user computer via a web server (as described above, for example). Similarly, a web server might receive web page requests and/or input data from a user computer and/or forward the web page requests and/or input data to an application server. In some cases a web server may be integrated with an application server.

In accordance with further embodiments, one or more servers 630 can function as a file server and/or can include one or more of the files (e.g., application code, data files, etc.) necessary to implement methods of the invention incorporated by an application running on a user computer and/or another server. Alternatively, as those skilled in the art will appreciate, a file server can include all necessary files, allowing such an application to be invoked remotely by a user computer and/or server. It should be noted that the functions described with respect to various servers herein (e.g., application server, database server, web server, file server, etc.) can be performed by a single server and/or a plurality of specialized servers, depending on implementation-specific needs and parameters.

In certain embodiments, the system can include one or more databases 640. The location of the database(s) 640 is discretionary. Merely by way of example, a database might reside on a storage medium local to (and/or resident in) a server (and/or a user computer). Alternatively, a database can be remote from any or all of the computers, so long as the database can be in communication (e.g., via the network) with one or more of these. In a particular set of embodiments, a database can reside in a storage-area network (“SAN”) familiar to those skilled in the art. (Likewise, any necessary files for performing the functions attributed to the computers can be stored locally on the respective computer and/or remotely, as appropriate.) In one set of embodiments, the database can be a relational database, such as an Oracle™ database, that is adapted to store, update, and retrieve data in response to SQL-formatted commands. The database might be controlled and/or maintained by a database server, as described above, for example.

While the invention has been particularly shown and described with reference to specific embodiments thereof, it will be understood by those skilled in the art that changes in the form and details of the disclosed embodiments may be made without departing from the spirit or scope of the invention. For example, embodiments have been described herein with reference to the use of conventional landlines and cellular phones. Additionally, the various embodiments of the invention as described may be implemented in the form of software running on a general purpose computer, in the form of a specialized hardware, or combination of software and hardware. It will be understood, however, that the invention is not so limited. That is, embodiments are contemplated in which a much wider diversity of communication devices may be employed in various combinations to effect redemption.

In addition, although various advantages, aspects, and objects of the present invention have been discussed herein with reference to various embodiments, it will be understood that the scope of the invention should not be limited by reference to such advantages, aspects, and objects. Rather, the scope of the invention should be determined with reference to the appended claims.

### EXAMPLES

### Example 1

### DNA Isolation

DNA from the collected saliva specimen was extracted using a standard DNA isolation protocol after a minimum of two days of storage at room temperature.

### Example 2

### DNA Quantification

Following DNA isolation, the human genomic DNA is in approximately 75 μL and a small portion of this DNA is quantified using a validated PicoGreen fluorescence assay protocol. The PicoGreen method uses fluorescence probes to detect the extracted human DNA. The amount of fluorescence is measured against a standardized concentration curve, corrected for background noise, and used to calculate the DNA concentration of each specimen. Extracted samples are either manually pipetted or automatically transferred to a Fluorotrac 200, 96-well plate for use on the BioTek Flx 800 Fluorometer.

### Example 3

### DNA Normalization and Integrity

DNA samples were normalized to 50 ng/μl (L-0052) and analyzed by gel QC according to using standard molecular biology methods. The plate of samples which have been quantified by the PicoGreen method and found to be at least 20 ng/μL are normalized using the BioMek® FX Liquid Handler. Samples measured to be greater than 200 ng/μL are diluted 1:10 with UltraPure Distilled Water into the acceptable range. Samples measured to be between 50 ng/μL and 200 ng/μL are normalized to a concentration of 50 ng/μL in this step. Samples measured to be between 20 and 50 ng/μL were unchanged in this step. Additionally, the quality of the DNA in the sample is evaluated based on gel electrophoresis. The DNAs passed gel QC (high molecular weight genomic DNA for integrity) and DNA quantification (>20 ng/μl) criteria and were tested in an 5-HTTLPR assays.

### Example 4

### Genotyping

CYP2C2D6 assays were designed using commercially synthesized nucleic acid primers and probes (Applied Biosystems (Carlsbad, Calif.)). All samples were genotyped for 5-HTTLPR assays on the Fluidigm system (EP1, BioMark, Biomark H D) (Fluidigm, San Francisco, Calif.) using Fluidigm's 96.96 dynamic arrays according to manufactures' standard procedures.

### Example 5

### Preamplification-Plate Set Up

A pooled assay mix is prepared by mixing the same primers of the PCR-based assays in the MedSelect DNA Insight Panel or other primers designed to scan the region targeted by the PCR-based assays. All of the primers amplifying the different genetic targets are multiplexed into one reaction. The pre-amplification step allows for the enrichment of genomic sequences. The pooled assay mix is combined with a commercial Multiplex PCR Master Mix (Qiagen) to prepare the Pre-Amp Master Mix.

The standard 96-well microtiter plates are set up for the pre-amplification step with the liquid handler placing 3.75 μL of Pre-Amp Master Mix into each well up to 95 wells for the run, leaving one well for the No Template control (NTC). Then the liquid handler adds 1.25 μL of gDNA from each patient specimen and 1.25 μL of the appropriate positive controls onto the plate. The microtiter plate is sealed and vortexed to ensure proper reagent mixing.

### Example 6

### Preamplification

PCR

Briefly, samples were amplified on a conventional PCR machine (14 cycles of 15 seconds at 95° C. and 4 minutes at 60° C.). This mixture was diluted 5-times; 2.5 μl were used for Fluidigm SNP genotyping application according to manufactures' standard procedures.

Results: Genotyping results were analyzed using an algorithm or system of algorithms, wherein the risk of patient use of one or more drugs based on the patient's genotype is assigned to categories such as one of the four categories below:

### 1. Use as Directed

### 2. Preferential Use

### 3. May Have Limitations or Significant Limitations

4. May Cause Serious Adverse Events Further output includes a text for each drug that is not assigned to the “Use as Directed” category (for Results see FIG. 8)

We claim:

1. A method for predicting an individual's likely response to a pain medication, comprising genotyping genetic variations in an individual to determine:
a categorical grade to an individual's likely ability to metabolize a particular pain medication and a categorical grade for a pain medication's potential efficacy with respect to the individual;
aggregating the categorical grades; and thereafter identifying the least positive grade as the recommended prediction for the individual.

2. The method of claim 1, further comprising genotyping genetic variations in the individual to determine a categorical grade for the individual to have a negative adverse reaction to the particular pain medication.

3. The method of any one of claims 1-2, wherein the pain medication is for chronic pain.

4. The method of any one of claims 1-3, wherein a genetic variation in the individual will reassign one or more of the categorical grades from a default category of typical use to preferential use or precautionary use.

5. The method of any one of claims 1-4, wherein a drug is prescribed to the individual with a recommendation of:
Use as directed
Preferential Use
Precautionary Use

6. The method of any one of claims 1-4, wherein each categorical grade is assigned to the three or more categories below:
Use as Directed
Preferential Use
May Have Limitations or Significant Limitations
May Cause Serious Adverse Events

7. The method of any one of claims 1-6, wherein the medication is a pain medication selected from acetaminophen, non-steroidal anti-inflammatory drug, corticosteroid, narcotic, or anti-convulsant.

8. The method of any one of claims 1-7, wherein the medication is a narcotic.

9. The method of any one of claims 1-8, wherein the narcotic is an opioid, opiate or opiate derivative.

10. The method of any one of claims 1-9, wherein the narcotic is selected from alfentanil, alphaprodine, anileridine, bezitramide, buprenorphine, butorphanol, codeine, dezocine, dihydrocodeine, diphenoxylate, ethylmorphine, fentanyl, heroin, hydrocodone, hydromorphone, isomethadone, levomethorphan, levorphanol, meptazinol, metazocine, metopon, morphine, nalbuphine, nalmefene, opium extracts, opium fluid extracts, pentazocine, propoxyphene, powdered opium, granulated opium, raw opium, tincture of opium, oxycodone, oxymorphone, pethidine(meperidine), phenazocine, piminodine, racemic methadone, racemethorphan, racemorphan, sufentanil, thebaine, or tramadol.

11. The method of any one of claims 1-10, wherein said method comprises genotyping a panel of at least one gene that affects the rate of drug metabolism and a panel of genes that affect a medication's potential efficacy with respect to the individual,

12. The method of any one of claims 1-11, wherein said method further comprises genotyping a panel of genes that affect the propensity for the individual to have a negative adverse reaction to a particular medication.

13. The method of any one of claims 1-12, wherein the panel for affecting drug metabolism comprises at least one gene that affects biochemical modification of pharmaceutical substances or xenobiotics and the panel for affecting efficacy comprises at least one opioid receptor modulating gene.

14. The method of any one of claims 1-13, wherein the panel for affecting adverse effect comprises at least one gene for undesired effects, e.g., side effects, that can be categorized as 1) mechanism based reactions and 2) idiosyncratic, “unpredictable” effects apparently unrelated to the primary pharmacologic action of the compound.

15. The method of any one of claims 1-14, wherein the panel of genes for affecting metabolism is at least one cytochrome P450 gene,

16. The method of any one of claims 1-15, wherein the panel for genes for affecting metabolism is at least two cytochrome P450 genes.

17. The method of any one of claims 1-16, wherein the panel of genes for affecting metabolism is at least one gene selected from CYP1A1, CYP2A6, CYP2C9, CYP2D6, CYP2E1, CYP3A5, CYP1A2, CYP1B1, CYP2B6, CYP2C8, CYP2C18, CYP2C19, CYP2E1, CYP3A4, CYP3A5, UGT1A4, UGT1A1, UGT1A9, UGT2B4, UGT2B7, UGT2B15, NAT1, NAT2, EPHX1, MTHFR, and ABCB1.

18. The method any one of claims 1-17, wherein the panel of genes for affecting efficacy is at least one gene for an opioid receptor gene.

19. The method of any one of claims 1-18, wherein the panel of genes for affecting efficacy a mu-opioid receptor gene.

20. The method of any one of claims 1-19, wherein the panel of genes for affecting drug metabolism is CYP2D6 and CYP2B6 genes, and wherein the panel of genes for affecting efficacy is the opioid receptor gene (OPRM1).

21. The method of any one of claims 1-20, wherein the panel of genes for affecting adverse reactions is selected from the serotonin receptor 2A (HTR2A), the serotonin gene 2C (HTR2C) and the major histocompatibility complex, class I, B (HLA-B).

22. The method of any one of claims 1-21, further comprising detecting a single nucleotide polymorphism in a gene of interest within each panel.

23. The method according to any one of claims 1-22, wherein said genotyping comprises analyzing a sample from the individual.

24. The method according to any one of claims 1-23, wherein said samples is selected from blood, including serum, lymphocytes, lymphoblastoid cells, fibroblasts, platelets, mononuclear cells or other blood cells, from saliva, liver, kidney, pancreas or heart, urine or from any other tissue, fluid, cell or cell line derived from the human body.

25. A computerized system for predicting an individual's likely response to a pain medication, comprising accessing the individual's genotype information, and
determining a categorical grade to an individual's likely ability to metabolize a particular pain medication and a categorical grade for a pain medication's potential efficacy with respect to the individual;
aggregating the categorical grades; and thereafter
identifying the least positive grade as the recommended prediction for the individual.

26. The computerized system of claim 25, wherein the system is accessed by healthcare providers.

27. The computerized system of any one of claims 25-26, wherein any potential conflicts and problems are flagged and displayed for the provider to review.

28. The computerized system of any one of claims 25-27, wherein a report is generated displaying recommendations for one or more medications.

29. The computerized system of any one of claims 25-28, wherein a genetic variation in the individual will reassign one or more of the categorical grades from a default category of typical use to preferential use or precautionary use.

30. The computerized system of any one of claims 25-29, wherein the pain medications is selected from acetaminophen, non-steroidal anti-inflammatory drug, corticosteroid, narcotic, or anti-convulsant.

31. The computerized system of any one of claims 25-30, wherein said genotyped information comprises a panel of at least one gene that affects the rate of drug metabolism and a panel of genes that affect a pain medication's potential efficacy with respect to the individual.

32. The computerized system of any one of claims 25-31, wherein said genotyped information further comprises a panel of genes that affect the propensity for the individual to have a negative adverse reaction to the particular pain medication.

33. A method of advising patient pain drug selection comprising the steps of
identifying a patent having a pain symptom to be addressed pharmaceutically,
identifying at least a drug to pharmaceutically address said pain symptom,
assaying genomic information of said patient,
evaluating the efficacy of said drug in view of said genetic information of said patient,
and providing to said patient a report evaluating said efficacy.

34. The method of claim 33 wherein said symptom is a symptom listed in FIG. 8.

35. The method of any one of claims 33-34 wherein said drug is a drug listed in FIG. 8.

36. The method of any one of claims 33-35 wherein said efficacy is an efficacy listed in FIG. 8.

37. The method of any one of claims 33-36 wherein said evaluating comprises placing a drug into a category.

38. The method of any one of claims 33-37, wherein said categorizing comprises placing said drug into one of four categories related to drug efficacy in view of patient genomic information.

39. The method of any one of claims 33-38, wherein said placing said drug into one of four categories comprises describing a drug as having ‘preferential use’, ‘use as directed,’ ‘significant limitations,’ or ‘serious adverse events.’

40. The method of any of claims 33-39, further comprising subjecting said report to a medical doctor's review prior to providing to said patient.
